(19) United States
(12) Patent Application Publication
(10) Pub. No.: US 2003/0008870 A1
(43) Pub. Date: Jan. 9, 2003

(54) Method of using a cyclooxygenase-2 inhibitor and sex steroids as a combination therapy for the treatment and prevention of dysmenorrhea

(76) Inventor: Joel Krasnow, Peapack, NJ (US)

Correspondence Address: Pharmacia Corporation, Corporate Patent Department, 800 North Lindbergh Blvd., Mail Zone O4E, St. Louis, MO, 63167, US

(21) Appl. No.: 10/067,128

(22) Filed: Feb. 4, 2002

Related U.S. Application Data
(60) Provisional application No. 60/266,261, filed on Feb. 2, 2001.

(51) Int. Cl. A61K 31/50; A61K 31/435; A61K 31/42; A61K 31/415

(52) U.S. Cl. 514/247; 514/277; 514/406; 514/378; 514/473

(57) ABSTRACT

The present invention provides methods for the treatment and prevention of dysmenorrhea in a woman using a combination of a cyclooxygenase-2 inhibitor and sex steroids.

### BACKGROUND OF THE INVENTION

1. Field of the Invention

The present invention relates to methods for the treatment and prevention of dysmenorrhea in a woman using a combination of a cyclooxygenase-2 inhibitor and sex steroids.

2. Description of the Related Art

In women, the menstrual cycle involves a complex series of hormonal changes. A consequence of these hormonal changes is the growth of the uterine lining (referred to as the endometrium). In the absence of pregnancy, the endometrium is shed in a process called menstruation. This process involves the release of prostaglandins, which cause contractions of the smooth muscle in the uterus. In some women, these contractions cause substantial pain, dysmenorrhea, which interferes with their daily activities.

The time at which menstruation occurs varies in that it can not be predicted with certainty in any one woman. The variability in the onset of menstrual cycles is dependent upon many variables including the individual woman, her age and underlying medical and psychosocial conditions. This makes it difficult to predict the onset of menses. Non-steroidal anti-inflammatory agents (NSAIDs) that inhibit prostaglandin synthesis are effective in reducing dysmenorrhea (Lundstrom, V., et al. Acta Obstet. Gynecol. Scand. Suppl., 113, 83-85 (1983)). They are most effective when administered prior to the onset of menstrual pain by 24-48 hours. Since predicting the precise timing of menstruation is difficult, attempts to maximize efficacy by initiating treatment prior to menses may result in several days of unnecessary medication.

The use of orally active contraceptives, composed of estrogen and progestin components, has been reported to reduce the intensity of the pain of dysmenorrhea (Nabrink, M. et al. Contraception, 42, 275-283 (1990)). The vast majority of oral contraceptives consist of a combination of a progestin sex steroid and an estrogen sex steroid. These sex steroids are administered concurrently for 21 days followed by either a 7 day pill free interval or by the administration of a placebo for 7 days in each 28 day cycle. Numerous regimens have been developed in which the progestin/estrogen combination is administered either as a fixed dosage combination (monophasic) or as a biphasic or a triphasic regimen in which the dosage of the combination is varied either once or twice throughout the menstrual cycle. Kuhl has reviewed the current state of hormonal contraception (Handb. Exp. Pharmacol., 135/II, 363-407 (1999)). Various oral contraceptive combinations are listed in WO 98/04265. Most current oral contraceptives give good menstrual cycle control (Thorneycroft, I. Am. J. Obstet. Gynecol., 180 (2, Pt. 2), S280-S287 (1999)).

When good relief of dysmenorrhea is not obtained through the use of oral contraceptives, a nonsteroidal anti-inflammatory drug can be added as treatment (Deligeoroglou, E. Annals of the New York Academy of Science, 900, 237-244 (2000)).

Prostaglandins play a major role in the inflammation process and the inhibition of prostaglandin production, especially production of PGG2, PGH2 and PGE2, has been a common target of anti-inflammatory drug discovery. However, common non-steroidal anti-inflammatory drugs (NSAIDs) that are active in reducing the prostaglandin-induced pain and swelling associated with the inflammation process are also active in affecting other prostaglandin-regulated processes not associated with the inflammation process. Thus, use of high doses of most common NSAIDs can produce severe side effects, including life-threatening ulcers, which limit their therapeutic potential. An alternative to NSAIDs is the use of corticosteroids, which have even more drastic side effects, especially when long-term therapy is involved.

Previous NSAIDs have been found to prevent the production of prostaglandins by inhibiting enzymes in the human arachidonic acid/prostaglandin pathway, including the enzyme cyclooxygenase (COX). The recent discovery of an inducible enzyme associated with inflammation (named “cyclooxygenase II (COX II)” or “prostaglandin G/H synthase II”) provides a viable target of inhibition that more effectively reduces inflammation and produces fewer and less drastic side effects.

U.S. Pat. No. 5,466,823 discloses pyrazolyl cyclooxygenase-2 inhibitors useful in treating inflammation and inflammation-related disorders, including menstrual cramps.

U.S. Pat. No. 5,932,598 discloses prodrugs of cyclooxygenase-2 inhibitors useful in treating inflammation and inflammation-related disorders, including menstrual cramps.

Morrison et al. describe a study where the cyclooxygenase-2 inhibitor, rofecoxib, is used to treat primary dysmenorrhea (Obstet. Gynecol., 94(4), 504-508 (1999)).

Compounds that selectively inhibit cyclooxygenase-2 and are useful in treating menstrual cramps have also been described in the following individual publications.

U.S. Pat. No. 5,521,207.

U.S. Pat. No. 5,633,272.

The various classes of compounds that are selective inhibitors of cyclooxygenase-2 have been reviewed by J. Talley in Prog. Med. Chem., 36, 201-234 (1999). Compounds that selectively inhibit cyclooxygenase-2 have also been described in the following individual publications.

U.S. Pat. No. 5,380,738.

U.S. Pat. No. 5,344,991.

U.S. Pat. No. 5,393,790.

U.S. Pat. No. 5,434,178.

U.S. Pat. No. 5,474,995.

U.S. Pat. No. 5,510,368.

WO 96/06840.

WO 96/03388.

WO 96/03387.

WO 96/19469.

WO 96/25405.

WO 95/15316.

WO 94/15932.

WO 94/27980.

WO 95/00501.

WO 94/13635.

WO 94/20480.

WO 94/26731.

The combination of NSAIDs and oral contraceptives has been used in cases where neither treatment alone was effective in treating primary dysmenorrhea (Coco, A., American Family Physician, 60(2), 489-496 (1999)).

U.S. Pat. No. 5,811,416 discloses the combination of an endothelin antagonist and/or an endothelin synthase inhibitor with at least one of a progestin, an estrogen, a combination of a progestin and estrogen, a cyclooxygenase inhibitor, a nitric oxide donor or a nitric oxide substrate for the treatment of menstrual disorders including dysmenorrhea.

U.S. Pat. No. 5,912,006 discloses the combination of an omega fatty acid and a cyclooxygenase inhibitor for the reduction or alleviation of uterine or vaginal pain associated with the onset of menstruation.

However, a combination therapy method for the treatment and prevention of dysmenorrhea comprising a COX-2 inhibitor and sex steroids has not been previously described.

### BRIEF SUMMARY OF THE INVENTION

To address the continuing need to find safe and effective agents for the prophylaxis and treatment of dysmenorrhea, combination therapies of therapeutic agents are now reported.

Among its several embodiments, the present invention provides a therapeutic combination of a cyclooxygenase-2 inhibitor compound source and an amount of sex steroid compounds, wherein the compounds together comprise a dysmenorrhea-effective amount of the compounds.

In another embodiment, the cyclooxygenase-2 inhibitor compound source is a cyclooxygenase-2 inhibitor compound.

In yet another embodiment, the present invention provides a combination therapy method for the treatment or prophylaxis of dysmenorrhea in a patient in need thereof comprising the use of an amount of a cyclooxygenase-2 inhibitor compound and an amount of a sex steroid, wherein the amounts of the cyclooxygenase-2 inhibitor compound and the sex steroid compound together comprise a dysmenorrhea-effective amount of the compounds.

The invention involves the preventive management of painful uterine cramps, dysmenorrhea, in women. A key improvement over existing technologies is that moderate to severe pain is not experienced prior to initiating treatment, but that it can be preempted, providing a much more satisfactory outcome. Another advantage is that by employing this regimen, lower doses of analgesic medication may be required. There should also be an advantage of a reduced blood loss compared with existing treatments.

Further scope of the applicability of the present invention will become apparent from the detailed description provided below. However, it should be understood that the following detailed description and examples, while indicating preferred embodiments of the invention, are given by way of illustration only since various changes and modifications within the spirit and scope of the invention will become apparent to those skilled in the art from this detailed description.

### DETAILED DESCRIPTION OF THE INVENTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference in their entirety.

Definitions

The following definitions are provided in order to aid the reader in understanding the detailed description of the present invention.

The phrase “cyclooxygenase-2 inhibitor” or “COX-2 inhibitor” or “cyclooxygenase-II inhibitor” includes agents that specifically inhibit a class of enzymes, cyclooxygenase-2, with less significant inhibition of cyclooxygenase-1.

Preferably, it includes compounds that have a cyclooxygenase-2 IC₅₀ of less than about 0.2 μM, and also have a selectivity ratio of cyclooxygenase-2 inhibition over cyclooxygenase-1 inhibition of at least 50, and more preferably of at least 100. Even more preferably, the compounds have a cyclooxygenase-1 IC₅₀ of greater than about 1 μM, and more preferably of greater than 10 μM.

The phrase “sex steroids” includes both estrogen and progestin steroid compounds.

The phrase “combination therapy” (or “co-therapy”) embraces the administration of a cyclooxygenase-2 inhibitor and a sex steroid as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies.

The phrase “therapeutically effective” is intended to qualify the combined amount of inhibitors in the combination therapy. This combined amount will achieve the goal of reducing or eliminating dysmenorrhea.

“Therapeutic compound” means a compound useful in the prophylaxis or treatment of dysmenorrhea.

The term “comprising” means “including the following elements but not excluding others.”

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical. Where used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl”, “lower alkenyl”, embrace radicals having “cis” and “trans” orientations, or alternatively, “E” and “Z” orientations.

The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl. The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl and cyclohexenyl.

The term “halo” means halogens such as fluorine, chlorine, bromine or iodine. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having one to six carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heterocyclo” embraces saturated, partially unsaturated and unsaturated heteroatom-containing ring-shaped radicals, where the heteroatoms may be selected from nitrogen, sulfur and oxygen. Examples of saturated heterocyclo radicals include saturated 3 to 6-membered heteromonocyclic groups containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclo radicals include dihydrothiophene, dihydropyran, dihydrofuran and dihydrothiazole.

The term “heteroaryl” embraces unsaturated heterocyclo radicals. Examples of unsaturated heterocyclo radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclo group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclo group containing 1 to 2 oxygen atoms and 1 to 3 nitrogen atoms (e.g. benzoxazolyl, benzoxadiazolyl, etc.); unsaturated 3 to 6-membered heteromonocyclic: group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclo group containing 1 to 2 sulfur atoms and 1 to 3 nitrogen atoms (e.g., benzothiazolyl, benzothiadiazolyl, etc.) and the like. The term also embraces radicals where heterocyclo radicals are fused with aryl radicals. Examples of such fused bicyclic radicals include benzofuran, benzothiophene, benzopyran, and the like. The terms benzopyran and chromene are interchangeable. Said “heterocyclo group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio. The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO₂. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The terms “sulfamyl”, “aminosulfonyl” and “sulfonamidyl” denote NH₂O₂S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl.

The term “carbonyl”, whether used alone or with other terms, such as “alkoxycarbonyl”, denotes —(C═O)—. The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO₂H. The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl. The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, haloalkyl and haloalkoxy.

The terms benzyl and phenylmethyl are interchangeable.

The term “heterocycloalkyl” embraces saturated and partially unsaturated heterocyclo-substituted alkyl radicals, such as pyrrolidinylmethyl, and heteroarylsubstituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “aralkoxy” embraces aralkyl radicals attached through an oxygen atom to other radicals. The term “aralkoxyalkyl” embraces aralkoxy radicals attached through an oxygen atom to an alkyl radical. The term “aralkylthio” embraces aralkyl radicals attached to a sulfur atom. The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like. The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like. The term “arylamino” denotes amino groups that have been substituted with one or two aryl radicals, such as N-phenylamino. The “arylamino” radicals may be further substituted on the aryl ring portion of the radical. The term “aralkylamino” embraces aralkyl radicals attached through an amino nitrogen atom to other radicals. The terms “N-arylaminoalkyl” and “N-aryl-N-alkylaminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH₂. The term “alkylaminocarbonyl” denotes an aminocarbonyl group that has been substituted with one or two alkyl radicals on the amino nitrogen atom. Preferred are “N-alkylaminocarbonyl” and “N,N-dialkylaminocarbonyl” radicals. More preferred are “lower N-alkylaminocarbonyl” and “lower N,N-dialkylaminocarbonyl” radicals with lower alkyl portions as defined above. The term “aminocarbonylalkyl” denotes a carbonylalkyl group that has been substituted with an amino radical on the carbonyl carbon atom.

The term “alkylaminoalkyl” embraces radicals having one or more alkyl radicals attached to an aminoalkyl radical. The term “aryloxyalkyl” embraces radicals having an aryl radical attached to an alkyl radical through a divalent oxygen atom. The term “arylthioalkyl” embraces radicals having an aryl radical attached to an alkyl radical through a divalent sulfur atom.

### Combinations

The methods and combinations of the present invention provide one or more benefits. Combinations of COX-2 inhibitors with the compounds, compositions, agents and therapies of the present invention are useful in treating and preventing dysmenorrhea. Preferably, the COX-2 inhibitors and the compounds, compositions, agents and therapies of the present invention are administered in combination at a low dose, that is, at a dose lower than has been conventionally used in clinical situations.

The combinations of the present invention will have a number of uses. For example, through dosage adjustment and medical monitoring, the individual dosages of the therapeutic compounds used in the combinations of the present invention will be lower than are typical for dosages of the therapeutic compounds when used in monotherapy. The dosage lowering will provide advantages including reduction of side effects of the individual therapeutic compounds when compared to the monotherapy. In addition, fewer side effects of the combination therapy compared with the monotherapies will lead to greater patient compliance with therapy regimens.

Alternatively, the methods and combination of the present invention can also maximize the therapeutic effect at higher doses.

When administered as a combination, the therapeutic agents can be formulated as separate compositions that are given at the same time or different times, or the therapeutic agents can be given as a single composition.

This new method of treatment for moderate to severe dysmenorrhea is superior to existing therapies, by reason of having the following characteristics. It inhibits the increased prostaglandin production induced by the complex series of hormonal changes characteristic of the menstrual cycle. The inhibition of prostaglandin synthesis occurs reproducibly 24-48 hours prior to initiation of menstruation. For safety reasons, it targets only the increased prostaglandin synthesis, which occurs immediately prior to menses, and not constitutive prostaglandin synthesis that may negatively impact other processes such as renal function.

The COX-2 enzyme, which is responsible for prostaglandin synthesis, has been demonstrated in the endometrium and myometrium of the uterus in women. The tissue distribution of COX-2 is significantly different from COX-1 in the endometrium. Therefore one would expect differences in the effects of COX-2 inhibitors compared to COX-1 inhibitors.

Among its several embodiments, the present invention provides a therapeutic combination of a cyclooxygenase-2 inhibitor compound source and a sex steroid compound, wherein the compounds together comprise a dysmenorrhea-effective amount of the compounds.

In another embodiment, the cyclooxygenase-2 inhibitor compound source is a cyclooxygenase-2 inhibitor compound.

In yet another embodiment, the cyclooxygenase-2 inhibitor compound source is a prodrug of a COX-2 inhibitor.

Nonlimiting examples of COX-2 inhibitors that may be used in the present invention are identified in Table 1 below.

TABLE 1
Cyclooxygenase-2 Inhibitors

| | Trade/ | | |
|---|---|---|---|
| | Research | | |
| Compound | Name | Reference | Dosage |
| 1,5-Diphenyl-3-substituted | | WO | |
| pyrazoles | | 97/13755 | |
| | radicicol | WO | |
| | | 96/25928. | |
| | | Kwon et al | |
| | | (Cancer | |
| | | Res (1992) | |
| | | 52 6296) | |
| | GB- | | |
| | 02283745 | | |
| | TP-72 | Cancer Res | |
| | | 1998 58 4 | |
| | | 717-723 | |
| 1-(4-chlorobenzoyl)-3-[4-(4- | A-183827.0 | | |
| fluoro-phenyl)thiazol-2- | | | |
| ylmethyl]-5-methoxy-2- | | | |
| methylindole | | | |
| | GR-253035 | | |
| 4-(4-cyclohexyl-2- | JTE-522 | JP 9052882 | |
| methyloxazol-5-yl)-2- | | | |
| fluorobenzenesulfonamide | | | |
| 5-chloro-3-(4- | | | |
| (methylsulfonyl)phenyl)-2- | | | |
| (methyl-5-pyridinyl)- | | | |
| pyridine | | | |
| 2-(3,5-difluoro-phenyl)-3-4- | | | |
| (methylsulfonyl)-phenyl)-2- | | | |
| cyclopenten-1-one | | | |
| | L-768277 | | |
| | L-783003 | | |
| | MK-966; | US 5968974 | 12.5-100 |
| | VIOXX ® | | mg po |
| indomethacin-derived | | WO | 200 |
| indolalkanoic acid | | 96/374679 | mg/kg/day |
| 1-Methylsulfonyl-4-[1,1- | | WO | |
| dimethyl-4-(4- | | 95/30656. | |
| fluorophenyl)cyclopenta-2,4- | | WO | |
| dien-3-yl]benzene | | 95/30652. | |
| | | WO | |
| | | 96/38418. | |
| | | WO | |
| | | 96/38442. | |
| 4,4-dimethyl-2-phenyl-3-[4- | | | |
| (methylsulfonyl)phenyl]cyclo- | | | |
| butenone | | | |
| 2-(4-methoxyphenyl)-4- | | EP 799823 | |
| methyl-1-(4- | | | |
| sulfamoylphenyl)-pyrrole | | | |
| N-[5-(4- | RWJ-63556 | | |
| fluoro)phenoxy]thiophene-2- | | | |
| methanesulfon-amide | | | |
| 5(E)-(3,5-di-tert-butyl-4- | S-2474 | EP 595546 | |
| hydroxy)benzylidene-2-ethyl- | | | |
| 1,2-isothiazolidine-1,1- | | | |
| dioxide | | | |
| 3-formylamino-7- | T-614 | DE | |
| methylsulfonylamino-6- | | 38/34204 | |
| phenoxy-4H-1-benzopyran-4- | | | |
| one | | | |
| Benzenesulfonamide, 4-(5-(4- | celecoxib | US 5466823 | |
| methylphenyl)-3- | | | |
| (trifluoromethyl)-1H- | | | |
| pyrazol-1-yl)- | | | |
| CS 502 | (Sankyo) | | |
| MK 633 | (Merck) | | |
| | meloxicam | US 4233299 | 15-30 |
| | | | mg/day |
| | nimesulide | US 3840597 | |

The following references listed in Table No. 2 below, hereby individually incorporated by reference, describe various COX-2 inhibitors suitable for use in the present invention described herein, and processes for their manufacture.

TABLE 2
COX-2 Inhibitor References

| | | | |
|---|---|---|---|
| WO 99/30721 | WO 99/30729 | US 5760068 | WO 98/15528 |
| WO 99/25695 | WO 99/24404 | WO 99/23087 | FR 27/71005 |
| EP 921119 | FR 27/70131 | WO 99/18960 | WO 99/15505 |
| WO 99/15503 | WO 99/14205 | WO 99/14195 | WO 99/14194 |
| WO 99/13799 | GB 23/30833 | US 5859036 | WO 99/12930 |
| WO 99/11605 | WO 99/10332 | WO 99/10331 | WO 99/09988 |
| US 5869524 | WO 99/05104 | US 5859257 | WO 98/47890 |
| WO 98/47871 | US 5830911 | US 5824699 | WO 98/45294 |
| WO 98/43966 | WO 98/41511 | WO 98/41864 | WO 98/41516 |
| WO 98/37235 | EP 86/3134 | JP 10/175861 | US 5776967 |
| WO 98/29382 | WO 98/25896 | ZA 97/04806 | EP 84/6,689 |
| WO 98/21195 | GB 23/19772 | WO 98/11080 | WO 98/06715 |
| WO 98/06708 | WO 98/07425 | WO 98/04527 | WO 98/03484 |
| FR 27/51966 | WO 97/38986 | WO 97/46524 | WO 97/44027 |
| WO 97/34882 | US 5681842 | WO 97/37984 | US 5686460 |
| WO 97/36863 | WO 97/40012 | WO 97/36497 | WO 97/29776 |
| WO 97/29775 | WO 97/29774 | WO 97/28121 | WO 97/28120 |
| WO 97/27181 | WO 95/11883 | WO 97/14691 | WO 97/13755 |
| WO 97/13755 | CA 21/80624 | WO 97/11701 | WO 96/41645 |
| WO 96/41626 | WO 96/41625 | WO 96/38418 | WO 96/37467 |
| WO 96/37469 | WO 96/36623 | WO 96/36617 | WO 96/31509 |
| WO 96/25405 | WO 96/24584 | WO 96/23786 | WO 96/19469 |
| WO 96/16934 | WO 96/13483 | WO 96/03385 | US 5510368 |
| WO 96/09304 | WO 96/06840 | WO 96/06840 | WO 96/03387 |
| WO 95/21817 | GB 22/83745 | WO 94/27980 | WO 94/26731 |
| WO 94/20480 | WO 94/13635 | FR 27/70,131 | US 5859036 |
| WO 99/01131 | WO 99/01455 | WO 99/01452 | WO 99/01130 |
| WO 98/57966 | WO 98/53814 | WO 98/53818 | WO 98/53817 |
| WO 98/47890 | US 5830911 | US 5776967 | WO 98/22101 |
| DE 19/753463 | WO 98/21195 | WO 98/16227 | US 5733909 |
| WO 98/05639 | WO 97/44028 | WO 97/44027 | WO 97/40012 |
| WO 97/38986 | US 5677318 | WO 97/34882 | WO 97/16435 |
| WO 97/03678 | WO 97/03667 | WO 96/36623 | WO 96/31509 |
| WO 96/25928 | WO 96/06840 | WO 96/21667 | WO 96/19469 |
| US 5510368 | WO 96/09304 | GB 22/83745 | WO 96/03392 |
| WO 94/25431 | WO 94/20480 | WO 94/13635 | JP 09052882 |
| GB 22/94879 | WO 95/15316 | WO 95/15315 | WO 96/03388 |
| WO 96/24585 | US 5344991 | WO 95/00501 | US 5968974 |
| US 5945539 | US 5994381 | | |

Three classes of cyclooxygenase-2 inhibitors are reviewed by J. Carter in Exp. Opin. Ther. Patents, 8(1), 21-29 (1997): methanesulfonanilides, tricyclics and structurally modified non-selective cyclooxygenase inhibitors. Methanesulfonanilides are a class of selective cyclooxygenase-2 inhibitors, of which NS-398, flosulide and nimesulide are example members.

A preferred class of tricyclic cyclooxygenase-2 inhibitors comprises compounds of formula (1)

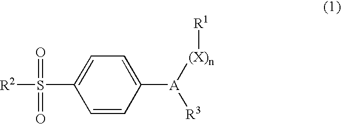

wherein A is a substituent selected from partially unsaturated or unsaturated heterocyclyl and partially unsaturated or unsaturated carbocyclic rings;

wherein n is 0 or 1;

wherein X is O or S;

wherein R¹ is at least one substituent selected from heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is methyl, amino or aminocarbonylalkyl; and

wherein R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl, wherein R³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; or a pharmaceutically-acceptable salt thereof.

Preferred COX-2 inhibitors are tricyclic COX-2 inhibitors wherein the A ring is selected from the heterocyclyl groups of pyrazolyl, furanonyl, isoxazolyl, pyridinyl and pyridazinonyl.

More preferred COX-2 inhibitors that may be used in the present invention include, but are not limited to:

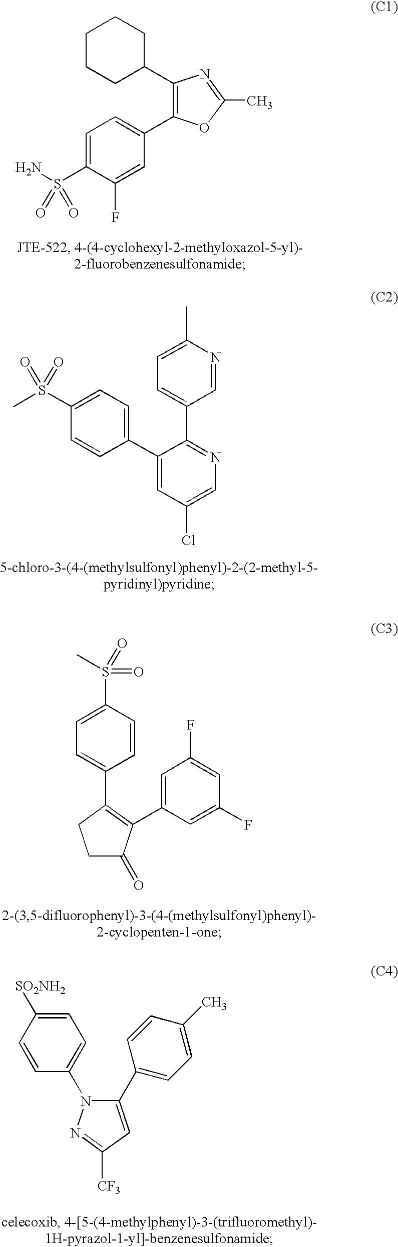

In a further preferred embodiment of the invention the cyclooxygenase inhibitor can be selected from the class of phenylacetic acid derivative cyclooxygenase-2 selective inhibitors represented by the general structure of Formula V:

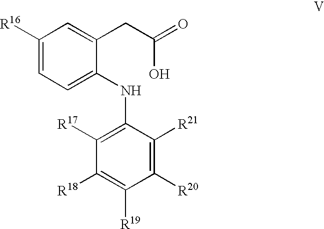

wherein

R¹⁶ is methyl or ethyl;

R¹⁷ is chloro or fluoro;

R¹⁸ is hydrogen or fluoro

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor that is described in WO 99/11605 is a compound that has the designation of COX189 (CAS RN 346670-74-4), and that has the structure shown in Formula V,

wherein

R¹⁶ is ethyl;

R¹⁷ and R¹⁹ are chloro;

R¹⁶ and R²⁰ are hydrogen; and

and R²¹ is methyl.

Other preferred cyclooxygenase-2 selective inhibitors that can be used in the present invention have the general structure shown in formula VI, where the J group is a carbocycle or a heterocycle. Particularly preferred embodiments have the structure:

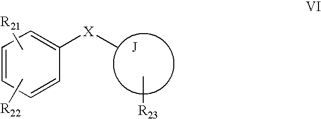

where:

X is O; J is 1-phenyl; R₂₁ is 2-NHSO₂CH₃; R₂₂ is 4-NO₂; and there is no R₂₃ group, (nimesulide), and

X is O; J is 1-oxo-inden-5-yl; R₂₁ is 2-F; R₂₂ is 4-F; and R₂₃ is 6-NHSO₂CH₃, (flosulide); and

X is O; J is cyclohexyl; R₂₁ is 2-NHSO₂CH₃; R₂₂ is 5-NO₂; and there is no R₂₃ group, (NS-398); and

X is S; J is 1-oxo-inden-5-yl; R₂₁ is 2-F; R₂₂ is 4-F; and R₂₃ is 6-N⁻SO₂CH₃.Na⁺, (L-745337); and

X is S; J is thiophen-2-yl; R₂₁ is 4-F; there is no R₂₂ group; and R₂₃ is 5-NHSO₂CH₃, (RWJ-63556); and

X is O; J is 2-oxo-5(R)-methyl-5-(2,2,2-trifluoroethyl)furan-(5H)-3-yl; R₂₁ is 3-F; R₂₂ is 4-F; and R₂₃ is 4-(p-SO₂CH₃)C₆H₄, (L-784512).

Further information on the applications of N-(2-cyclohexyloxynitrophenyl)methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J.-P. et al., in Science Spectra, available at: http://www.gbhap.com/Science_Spectra/20-1-article.htm

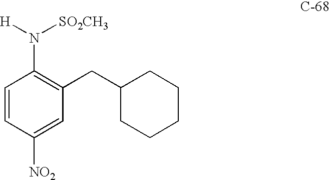

(Jun. 6, 2001); and Iwata, K. et al., in Jpn. J. Pharmacol., 75(2) :191-194 (1997).

An evaluation of the antiinflammatory activity of the cyclooxygenase-2 selective inhibitor, RWJ 63556, in a canine model of inflammation, was described by Kirchner et al., in J Pharmacol Exp Ther 282, 1094-1101 (1997).

Other compounds useful as the cyclooxygenase-2 selective inhibitor in the present invention include diarylmethylidenefuran derivatives such as those described in U.S. Pat. No. 6,180,651. Such diarylmethylidenefuran derivatives have the general formula shown below in formula VII:

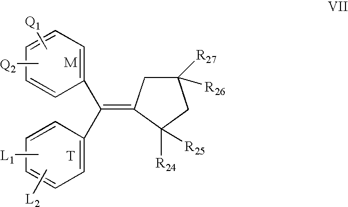

wherein:

the rings T and M independently are:

a phenyl radical,

a naphthyl radical,

a radical derived from a heterocycle comprising 5 to 6 members and possessing from 1 to 4 heteroatoms, or a radical derived from a saturated hydrocarbon ring having from 3 to 7 carbon atoms;

at least one of the substituents Q₁, Q₂, L₁ or L₂ is:

an —S(O)ₙ—R group, in which n is an integer equal to 0, 1 or 2 and R is a lower alkyl radical having 1 to 6 carbon atoms or a lower haloalkyl radical having 1 to 6 carbon atoms, or

an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q₁ and Q₂ or L₁ and L₂ are a methylenedioxy group; and

R₂₄, R₂₅, R₂₆ and R₂₇ independently are:

a hydrogen atom,

a halogen atom,

a lower alkyl radical having 1 to 6 carbon atoms,

a lower haloalkyl radical having 1 to 6 carbon atoms, or

an aromatic radical selected from the group consisting of phenyl, naphthyl, thienyl, furyl and pyridyl; or,

R₂₄, R₂₅ or R₂₆, R₂₇ are an oxygen atom, or

R₂₄, R₂₅ or R₂₆, R₂₇, together with the carbon atom to which they are attached, form a saturated hydrocarbon ring having from 3 to 7 carbon atoms;

or an isomer or prodrug thereof.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl] benzenesulfonamide.

Preferred cyclooxygenase-2 selective inhibitors that are useful in the present invention include the following individual compounds; darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

In another preferred embodiment of the invention, the compound BMS-347070 having the formula:

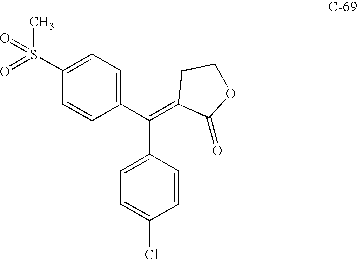

Information about S-33516, mentioned above, can be found in Current Drugs Headline News, at http://www.current-drugs.com/NEWS/Inflam1.htm, Oct. 4, 2001, where it was reported that S-33516 is a tetrahydroisoinde derivative which has IC₅₀ values of 0.1 and 0.001 mM against cyclooxygenase-1 and cyclooxygenase-2, respectively. In human whole blood, S-33516 was reported to have an ED₅₀=0.39 mg/kg.

The CAS reference numbers for nonlimiting examples of COX-2 inhibitors are identified in Table 3 below.

TABLE 3
COX-2 Inhibitors

| Compound Number | CAS Reference Number |
|---|---|
| C1 | 180200-68-4 |
| C2 | 202409-33-4 |
| C3 | 212126-32-4 |
| C4 | 169590-42-5 |
| C5 | 162011-90-7 |
| C6 | 181695-72-7 |
| C7 | 198470-84-7 |
| C8 | 170569-86-5 |
| C9 | 187845-71-2 |
| C10 | 179382-91-3 |
| C11 | 51803-78-2 |
| C12 | 189954-13-0 |
| C13 | 158205-05-1 |
| C14 | 197239-99-9 |
| C15 | 197240-09-8 |
| C16 | 226703-01-1 |
| C17 | 93014-16-5 |
| C18 | 197239-97-7 |
| C19 | 162054-19-5 |
| C20 | 170569-87-6 |
| C21 | 279221-13-5 |
| C22 | 170572-13-1 |
| C23 | 123653-11-2 |
| C24 | 80937-31-1 |
| C25 | 279221-14-6 |
| C26 | 279221-15-7 |
| C27 | 187846-16-8 |
| C28 | 189954-16-3 |
| C29 | 181485-41-6 |
| C30 | 187845-80-3 |
| C31 | 158959-32-1 |
| C32 | 170570-29-3 |
| C33 | 177660-77-4 |
| C34 | 177660-95-6 |
| C35 | 181695-81-8 |
| C36 | 197240-14-5 |
| C37 | 181696-33-3 |
| C38 | 178816-94-9 |
| C39 | 178816-61-0 |
| C40 | 279221-17-9 |
| C41 | 187845-71-2 |
| C42 | 123663-49-0 |
| C43 | 197905-01-4 |
| C44 | 197904-84-0 |
| C45 | 169590-41-4 |
| C46 | 202409-33-4 |
| C47 | 88149-94-4 |
| C48 | 266320-83-6 |
| C49 | 215122-43-3 |
| C50 | 215122-44-4 |
| C51 | 215122-74-0 |
| C52 | 215123-80-1 |
| C53 | 215122-70-6 |
| C54 | 264878-87-7 |
| C55 | 279221-12-4 |
| C56 | 215123-48-1 |
| C57 | 215123-03-8 |
| C58 | 215123-60-7 |
| C59 | 279221-18-0 |
| C60 | 215123-61-8 |
| C61 | 215123-52-7 |
| C62 | 279221-19-1 |
| C63 | 215123-64-1 |
| C64 | 215123-70-9 |
| C65 | 215123-79-8 |
| C66 | 215123-91-4 |
| C67 | 215123-77-6 |

More preferably, the COX-2 inhibitors that may be used in the present invention include, but are not limited to celecoxib, valdecoxib, parecoxcib, rofecoxib, NS-398, deracoxib, Merck MK-663 and ABT-963.

Various classes of cyclooxygenase-2 inhibitors can be prepared as follows. Pyrazoles can be prepared by methods described in WO 95/15316. Pyrazoles can further be prepared by methods described in WO 95/15315. Pyrazoles can also be prepared by methods described in WO 96/03385. Thiophene analogs can be prepared by methods described in WO 95/00501. Preparation of thiophene analogs is also described in WO 94/15932. Oxazoles can be prepared by the methods described in WO 95/00501. Preparation of oxazoles is also described in WO 94/27980. Isoxazoles can be prepared by the methods described in WO 96/03388. Preparation of imidazoles is also described in WO 96/03387. Cyclopentene cyclooxygenase-2 inhibitors can be prepared by the methods described in U.S. Pat. No. 5,344,991. Preparation of cyclopentene COX-2 inhibitors is also described in WO 95/00501. Terphenyl compounds can be prepared by the methods described in WO 96/03392. Preparation of pyridine compounds is also described in WO 96/24,585. Benzopyranopyrazolyl compounds can be prepared by the methods described in WO 96/09304. Benzopyran compounds can be prepared by the methods described in WO 98/47890. Preparation of benzopyran compounds is also described in WO 00/23433. Benzopyran compounds can further be prepared by the methods described in U.S. Pat. No. 6,077,850. Preparation of benzopyran compounds is further described in U.S. Pat. No. 6,034,256. Arylpyridazinones can be prepared by the methods described in WO 00/24719.

The celecoxib used in the therapeutic combinations of the present invention can be prepared in the manner set forth in U.S. Pat. No. 5,466,823.

The valdecoxib used in the therapeutic combinations of the present invention can be prepared in the manner set forth in U.S. Pat. No. 5,633,272.

The parecoxib used in the therapeutic combinations of the present invention can be prepared in the manner set forth in U.S. Pat. No. 5,932,598.

The rofecoxib used in the therapeutic combinations of the present invention can be prepared in the manner set forth in U.S. Pat. No. 5,474,995.

The deracoxib used in the therapeutic combinations of the present invention can be prepared in the manner set forth in U.S. Pat. No. 5,521,207.

The compound MK-663 used in the therapeutic combinations of the present invention can be prepared in the manner set forth in WO 98/03484.

The compound NS-398 used in the therapeutic combinations of the present invention can be prepared in the manner set forth in U.S. Pat. No. 4,885,367.

The compound ABT-963 used in the therapeutic combinations of the present invention can be prepared in the manner set forth in WO 00/24719.

The estrogen sex steroid is preferably selected from, but is not limited to, the group consisting of ethinyl estradiol, 17β-estradiol and mestranol.

Still more preferably the estrogen sex steroid is ethinyl estradiol.

The progestin sex steroid is preferably selected from, but is not limited to, the group consisting of levonorgestrel, norethindrone acetate, norgestimate, ethynodiol acetate, desogestrel, norgestrel, gestodene, 3-ketodesogestrel, Org 30659, dienogest, trimegestone and norethindrone.

More preferably the progestin sex steroid is selected from the group consisting of levonorgestrel, norethindrone acetate, norgestimate, ethynodiol acetate, desogestrel, norgestrel and norethindrone.

Even more preferably, the progestin sex steroid is selected from the group consisting of levonorgestrel, norethindrone acetate and norgestimate.

The structures and CAS registry numbers of preferred estrogen and progestin sex steroids are listed in Table No. 4 below.

TABLE 4
Sex Steroid Structures

| | CAS | |
|---|---|---|
| | Registry | |
| Name | Number | Structure |
| Ethinyl estradiol | 57-93-6 | 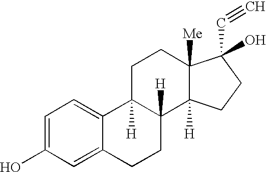 |
| 17β-Estradiol | 50-28-2 | 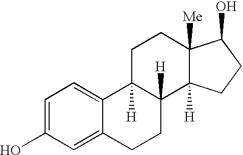 |
| Mestranol | 72-33-3 | 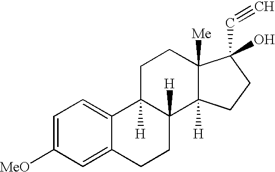 |
| Levonorgestrel | 797-63-7 | 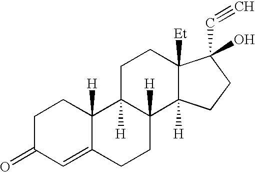 |
| Norethindrone acetate | 51-98-9 | 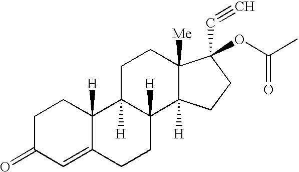 |
| Norgestimate | 35189-28-7 | 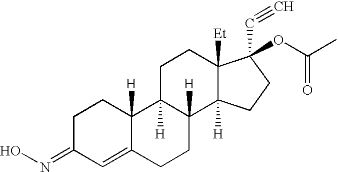 |
| Ethynodiol diacetate | 297-76-7 | 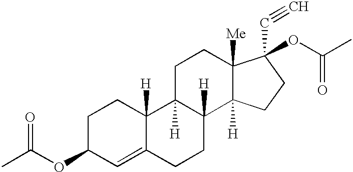 |
| Desogestrel | 54024-22-5 | 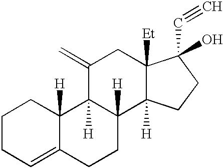 |
| Norgestrel | 6533-00-2 | 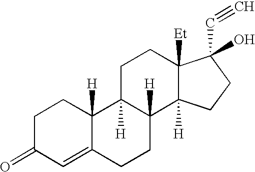 |
| Norethindrone | 68-22-4 | 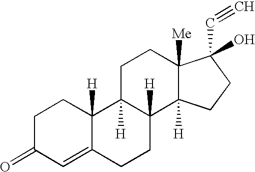 |
| 3-Ketodesogestrel | 54048-10-1 | 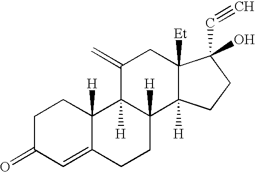 |
| Gestodene | 60282-87-3 | 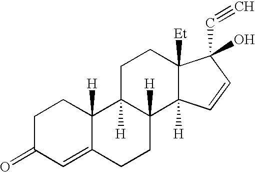 |
| Org 30659 | 110072-15-6 | 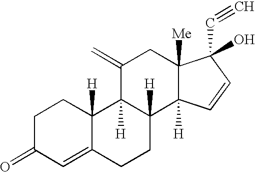 |
| Trimegestone | 74513-62-5 | 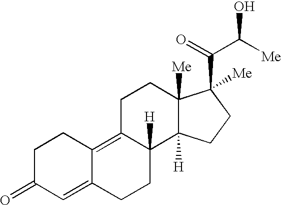 |
| Dienogest | 65928-58-7 | 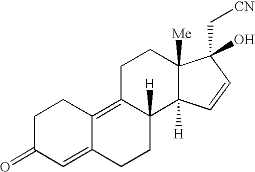 |

The following references listed in Table No. 5 below, hereby individually incorporated by reference, describe various sex steroids suitable for use in the present invention described herein, and processes for their manufacture.

TABLE 5
Sex Steroid References

| Sex Steroid | Reference |
|---|---|
| Ethinyl estradiol | U.S. Pat. No. 3,759,961 |
| 17β-Estradiol | U.S. Pat. No. 3,274,182 |
| Mestranol | U.S. Pat. No. 3,759,961 |
| Levonorgestrel | U.S. Pat. No. 3,759,961 |
| Norethindrone acetate | U.S. Pat. No. 3,408,371 |
| Norgestimate | U.S. Pat. No. 4,027,019 |
| Ethynodiol diacetate | U.S. Pat. No. 3,383,384 |
| Desogestrel | U.S. Pat. No. 3,927,046 |
| Norgestrel | U.S. Pat. No. 3,892,779 |
| Norethindrone | U.S. Pat. No. 3,383,384 |
| 3-Ketodesogestrel | U.S. Pat. No. 4,371,529 |
| Gestodene | U.S. Pat. No. 4,081,537 |
| Org 30659 | U.S. Pat. No. 5,236,913 |
| Trimegestone | U.S. Pat. No. 4,273,771 |
| Dienogest | U.S. Pat. No. 4,167,517 |

The compounds useful in the present invention can have no asymmetric carbon atoms, or, alternatively, the useful compounds can have one or more asymmetric carbon atoms. When the useful compounds have one or more asymmetric carbon atoms, they therefore include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention.

The compounds useful in the present invention also include tautomers.

The compounds useful in the present invention also include their salts, solvates and prodrugs.

Dosages, Formulations and Routes of Administration

For the prophylaxis or treatment of the conditions referred to above, the compounds useful in the combinations and methods of the present invention can be used as the compound per se. Pharmaceutically acceptable salts are particularly suitable for medical applications because of their greater aqueous solubility relative to the parent compound. Such salts must clearly have a pharmaceutically acceptable anion or cation. Suitable pharmaceutically acceptable acid addition salts of the compounds of the present invention when possible include those derived from inorganic acids, such as hydrochloric, hydrobromic, phosphoric, metaphosphoric, nitric, sulfonic, and sulfuric acids, and organic acids such as formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Suitable pharmaceutically-acceptable base addition salts of compounds of the present invention include metallic ion salts and organic ion salts. More preferred metallic ion salts include, but are not limited to appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The compounds useful in the present invention can be presented with an acceptable carrier in the form of a pharmaceutical composition. The carrier must, of course, be acceptable in the sense of being compatible with the other ingredients of the composition and must not be deleterious to the recipient. The carrier can be a solid or a liquid, or both, and is preferably formulated with the compound as a unit-dose composition, for example, a tablet, which can contain from 0.05% to 95% by weight of the active compound. Other pharmacologically active substances can also be present, including other compounds of the present invention. The pharmaceutical compositions of the invention can be prepared by any of the well-known techniques of pharmacy, consisting essentially of admixing the components.

Optionally, the combination of the present invention can comprise a composition comprising a cyclooxygenase-2 inhibiting compound and a sex steroid compound. In such a composition, the cyclooxygenase-2 inhibiting compound and the sex steroid can be present in a single dosage form, for example a pill, a capsule, or a liquid that contains both of the compounds.

These compounds can be administered by any conventional means available for use in conjunction with pharmaceuticals, either as individual therapeutic compounds or as a combination of therapeutic compounds.

The amount of compound which is required to achieve the desired biological effect will, of course, depend on a number of factors such as the specific compound chosen, the use for which it is intended, the mode of administration, and the clinical condition of the recipient.

### Dosages

Dosage levels of COX-2 inhibitors on the order of about 0.1 mg to about 10,000 mg of the active ingredient compound are useful in the treatment of the above conditions, with preferred levels of about 1.0 mg to about 1,000 mg and even more preferred levels of about 5 mg to about 500 mg. The amount of active ingredient will vary depending upon the host treated and the particular mode of administration.

It is understood, however, that a specific dose level for any particular patient will depend upon a variety of factors including the activity of the specific compound employed, the age, body weight, general health, sex, diet, time of administration, rate of excretion, drug combination, and the severity of the particular disease being treated and form of administration.

Treatment dosages generally may be titrated to optimize safety and efficacy. Typically, dosage-effect relationships from in vitro initially can provide useful guidance on the proper doses for patient administration. Studies in animal models also generally may be used for guidance regarding effective dosages for treatment of cancers in accordance with the present invention. In terms of treatment protocols, it should be appreciated that the dosage to be administered will depend on several factors, including the particular agent that is administered, the route administered, the condition of the particular patient, etc. Generally speaking, one will desire to administer an amount of the compound that is effective to achieve a serum level commensurate with the concentrations found to be effective in vitro. Thus, where a compound is found to demonstrate in vitro activity at, e.g., 10 μM, one will desire to administer an amount of the drug that is effective to provide about a 10 μM concentration in vivo. Determination of these parameters is well within the skill of the art. These considerations, as well as effective formulations and administration procedures are well known in the art and are described in standard textbooks.

An estrogen sex steroid at a daily dosage equivalent in estrogenic activity to about 5-75 ug ethinyl estradiol is useful in the treatment of the above conditions, with preferred levels of about 10 ug to about 50 ug and even more preferred levels of about 15 ug to about 35 ug. Actual dosage levels for other estrogen sex steroids may vary relative to the levels listed for ethinyl estradiol. A progestin sex steroid at a daily dosage equivalent in progestinic activity to about 10-600 ug levonorgestrel is useful in the treatment of the above conditions, with preferred levels of about 25 ug to about 400 ug and even more preferred levels of about 50 ug to about 200 ug. Actual dosage levels for other progestin sex steroids may vary relative to the levels listed for levonorgestrel.

The compounds of the present invention can be formulated as a pharmaceutical composition. Such a composition can then be administered orally, parenterally, by inhalation spray, rectally, or topically in dosage unit formulations containing conventional nontoxic pharmaceutically acceptable carriers, adjuvants, and vehicles as desired. Formulation of drugs is discussed in, for example, Hoover, John E., Remington's Pharmaceutical Sciences, Mack Publishing Co., Easton, Pa. 1975. Another discussion of drug formulations can be found in Liberman, H. A. and Lachman, L., Eds., Pharmaceutical Dosage Forms, Marcel Decker, New York, N.Y., 1980.

Solid dosage forms for oral administration can include capsules, tablets, pills, powders, and granules. In such solid dosage forms, the compounds of this invention are ordinarily combined with one or more adjuvants appropriate to the indicated route of administration. If administered per os, a contemplated inhibitor compound can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The term parenteral as used herein includes subcutaneous injections, intravenous, intramuscular, intrasternal injection, or infusion techniques. Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. A contemplated therapeutic compound can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Suppositories for rectal administration of the drug can be prepared by mixing the drug with a suitable nonirritating excipient such as cocoa butter, synthetic mono- di- or triglycerides, fatty acids and polyethylene glycols that are solid at ordinary temperatures but liquid at the rectal temperature and will therefore melt in the rectum and release the drug.

Topical administration can also involve the use of transdermal administration such as transdermal patches or iontophoresis devices.

The amount of active ingredient that can be combined with the carrier materials to produce a single dosage form varies depending upon the mammalian host treated and the particular mode of administration.

### Treatment Regimen

The dosage regimen to prevent, give relief from, or ameliorate a disease condition having dysmenorrhea as an element of the disease or to protect against or treat a further dysmenorrhea related disorder with the compounds and/or compositions of the present invention is selected in accordance with a variety of factors. These include the type, age, weight, diet, and medical condition of the patient, the severity of the disease, the route of administration, pharmacological considerations such as the activity, efficacy, pharmacokinetics and toxicology profiles of the particular compound employed, whether a drug delivery system is utilized, and whether the compound is administered as part of a drug combination. Thus, the dosage regimen actually employed may vary widely and therefore deviate from the preferred dosage regimen set forth above.

In order to create a reproducible time of menses, specific combinations of daily administration of orally active sex steroids will be used to pharmacologically regulate the onset of menses within a small (24-48 hour) window of time. These steroids will include an estrogenic component and a progestagenic component with the effects of the latter predominating. The use of such a regimen should also result in less growth of the endometrial lining resulting in a reduced blood loss at the time of menses.

The use of daily orally active sex steroids to regulate endometrial growth will upon their discontinuation result in menses within 48-72 hours. The addition of a cyclooxygenase-2 inhibitor, such as celecoxib, starting 24 hours following discontinuation of the sex steroids will synchronize events such that the cyclooxygenase-2 inhibitor will be reproducibly administered at the time of initiation of increased prostaglandin synthesis triggered by the withdrawal of the steroid hormones. The cyclooxgenase-2 inhibitor can be administered until the end of menses with a variety of regimens. For example, the cyclooxygenase-2 inhibitor can be administered daily (od), twice a day (bid) or three times a day (tid). Thus the invention refers to the sequential administration of daily orally active sex steroids followed by a selective COX-2 inhibitor. This would be administered in a regular schedule (every 28 days) with the sex steroids being administered for 21 days followed by 2-7 days of a cyclooxygenase-2 inhibitor. More preferably, the sex steroids would be administered for 21 days followed by 4-7 days of a cyclooxygenase-2 inhibitor.

Patients undergoing treatment with the compounds or compositions disclosed herein can be routinely monitored to determine the effectiveness of the combination therapy. Continuous analysis of such data permits modification of the treatment regimen during therapy so that optimal effective amounts of each type of therapeutic compound are administered at any point in time, and so that the duration of treatment can be determined as well. In this way, the treatment regimen/dosing schedule can be rationally modified over the course of therapy so that the lowest amount of the therapeutic compounds which together exhibit satisfactory effectiveness is administered, and so that administration is continued only so long as is necessary to successfully treat the dysmenorrhea related condition.

A potential advantage of the combination therapy disclosed herein may be reduced dosage amount of any individual therapeutic compound, or all therapeutic compounds, effective in treating dysmenorrhea related conditions. The dosage lowering will provide advantages including reduction of side effects of the individual therapeutic compounds when compared to the monotherapy.

One of the several embodiments of the present invention provides a combination therapy comprising the use of a first amount of a COX-2 inhibitor and a second amount of sex steroids useful in the prophylaxis or treatment of dysmenorrhea, wherein said first and second amounts together comprise an dysmenorrhea-effective amount of said compounds. For example one of the many embodiments of the present invention is a combination therapy regimen comprising therapeutic dosages of a pyrazole COX-2 inhibitor, ethinyl estradiol and levonorgestrel.

The following non-limiting examples serve to illustrate various aspects of the present invention.

### EXAMPLES

Table 6 illustrates examples of some combinations of the present invention wherein the combination comprises a first amount of a COX-2 inhibitor source, a second amount of a estrogen sex steroid and a third amount of a progestin sex steroid wherein the amounts together comprise an dysmenorrhea-effective amount of the compounds.

TABLE 6
Combination Examples

| Example | COX-2 | | |
|---|---|---|---|
| Number | Inhibitor | Estrogen Sex Steroid | Progestin Sex Steroid |
| 1 | C1 | Ethinyl estradiol | Levonorgestrel |
| 2 | C2 | Ethinyl estradiol | Levonorgestrel |
| 3 | C3 | Ethinyl estradiol | Levonorgestrel |
| 4 | C4 | Ethinyl estradiol | Levonorgestrel |
| 5 | C5 | Ethinyl estradiol | Levonorgestrel |
| 6 | C6 | Ethinyl estradiol | Levonorgestrel |
| 7 | C7 | Ethinyl estradiol | Levonorgestrel |
| 8 | C8 | Ethinyl estradiol | Levonorgestrel |
| 9 | C9 | Ethinyl estradiol | Levonorgestrel |
| 10 | C10 | Ethinyl estradiol | Levonorgestrel |
| 11 | C11 | Ethinyl estradiol | Levonorgestrel |
| 12 | C12 | Ethinyl estradiol | Levonorgestrel |
| 13 | C13 | Ethinyl estradiol | Levonorgestrel |
| 14 | C14 | Ethinyl estradiol | Levonorgestrel |
| 15 | C15 | Ethinyl estradiol | Levonorgestrel |
| 16 | C16 | Ethinyl estradiol | Levonorgestrel |
| 17 | C17 | Ethinyl estradiol | Levonorgestrel |
| 18 | C18 | Ethinyl estradiol | Levonorgestrel |
| 19 | C19 | Ethinyl estradiol | Levonorgestrel |
| 20 | C20 | Ethinyl estradiol | Levonorgestrel |
| 21 | C21 | Ethinyl estradiol | Levonorgestrel |
| 22 | C22 | Ethinyl estradiol | Levonorgestrel |
| 23 | C23 | Ethinyl estradiol | Levonorgestrel |
| 24 | C24 | Ethinyl estradiol | Levonorgestrel |
| 25 | C25 | Ethinyl estradiol | Levonorgestrel |
| 26 | C26 | Ethinyl estradiol | Levonorgestrel |
| 27 | C27 | Ethinyl estradiol | Levonorgestrel |
| 28 | C28 | Ethinyl estradiol | Levonorgestrel |
| 29 | C29 | Ethinyl estradiol | Levonorgestrel |
| 30 | C30 | Ethinyl estradiol | Levonorgestrel |
| 31 | C31 | Ethinyl estradiol | Levonorgestrel |
| 32 | C32 | Ethinyl estradiol | Levonorgestrel |
| 33 | C33 | Ethinyl estradiol | Levonorgestrel |
| 34 | C34 | Ethinyl estradiol | Levonorgestrel |
| 35 | C35 | Ethinyl estradiol | Levonorgestrel |
| 36 | C36 | Ethinyl estradiol | Levonorgestrel |
| 37 | C37 | Ethinyl estradiol | Levonorgestrel |
| 38 | C38 | Ethinyl estradiol | Levonorgestrel |
| 39 | C39 | Ethinyl estradiol | Levonorgestrel |
| 40 | C40 | Ethinyl estradiol | Levonorgestrel |
| 41 | C41 | Ethinyl estradiol | Levonorgestrel |
| 42 | C42 | Ethinyl estradiol | Levonorgestrel |
| 43 | C43 | Ethinyl estradiol | Levonorgestrel |
| 44 | C44 | Ethinyl estradiol | Levonorgestrel |
| 45 | C45 | Ethinyl estradiol | Levonorgestrel |
| 46 | C46 | Ethinyl estradiol | Levonorgestrel |
| 47 | C47 | Ethinyl estradiol | Levonorgestrel |
| 48 | C48 | Ethinyl estradiol | Levonorgestrel |
| 49 | C49 | Ethinyl estradiol | Levonorgestrel |
| 50 | C50 | Ethinyl estradiol | Levonorgestrel |
| 51 | C51 | Ethinyl estradiol | Levonorgestrel |
| 52 | C52 | Ethinyl estradiol | Levonorgestrel |
| 53 | C53 | Ethinyl estradiol | Levonorgestrel |
| 54 | C54 | Ethinyl estradiol | Levonorgestrel |
| 55 | C55 | Ethinyl estradiol | Levonorgestrel |
| 56 | C56 | Ethinyl estradiol | Levonorgestrel |
| 57 | C57 | Ethinyl estradiol | Levonorgestrel |
| 58 | C58 | Ethinyl estradiol | Levonorgestrel |
| 59 | C59 | Ethinyl estradiol | Levonorgestrel |
| 60 | C60 | Ethinyl estradiol | Levonorgestrel |
| 61 | C61 | Ethinyl estradiol | Levonorgestrel |
| 62 | C62 | Ethinyl estradiol | Levonorgestrel |
| 63 | C63 | Ethinyl estradiol | Levonorgestrel |
| 64 | C64 | Ethinyl estradiol | Levonorgestrel |
| 65 | C65 | Ethinyl estradiol | Levonorgestrel |
| 66 | C66 | Ethinyl estradiol | Levonorgestrel |
| 67 | C67 | Ethinyl estradiol | Levonorgestrel |
| 68 | C1 | Ethinyl estradiol | Norethindrone acetate |
| 69 | C2 | Ethinyl estradiol | Norethindrone acetate |
| 70 | C3 | Ethinyl estradiol | Norethindrone acetate |
| 71 | C4 | Ethinyl estradiol | Norethindrone acetate |
| 72 | C5 | Ethinyl estradiol | Norethindrone acetate |
| 73 | C6 | Ethinyl estradiol | Norethindrone acetate |
| 74 | C7 | Ethinyl estradiol | Norethindrone acetate |
| 75 | C8 | Ethinyl estradiol | Norethindrone acetate |
| 76 | C9 | Ethinyl estradiol | Norethindrone acetate |
| 77 | C10 | Ethinyl estradiol | Norethindrone acetate |
| 78 | C11 | Ethinyl estradiol | Norethindrone acetate |
| 79 | C12 | Ethinyl estradiol | Norethindrone acetate |
| 80 | C13 | Ethinyl estradiol | Norethindrone acetate |
| 81 | C14 | Ethinyl estradiol | Norethindrone acetate |
| 82 | C15 | Ethinyl estradiol | Norethindrone acetate |
| 83 | C16 | Ethinyl estradiol | Norethindrone acetate |
| 84 | C17 | Ethinyl estradiol | Norethindrone acetate |
| 85 | C18 | Ethinyl estradiol | Norethindrone acetate |
| 86 | C19 | Ethinyl estradiol | Norethindrone acetate |
| 87 | C20 | Ethinyl estradiol | Norethindrone acetate |
| 88 | C21 | Ethinyl estradiol | Norethindrone acetate |
| 89 | C22 | Ethinyl estradiol | Norethindrone acetate |
| 90 | C23 | Ethinyl estradiol | Norethindrone acetate |
| 91 | C24 | Ethinyl estradiol | Norethindrone acetate |
| 92 | C25 | Ethinyl estradiol | Norethindrone acetate |
| 93 | C26 | Ethinyl estradiol | Norethindrone acetate |
| 94 | C27 | Ethinyl estradiol | Norethindrone acetate |
| 95 | C28 | Ethinyl estradiol | Norethindrone acetate |
| 96 | C29 | Ethinyl estradiol | Norethindrone acetate |
| 97 | C30 | Ethinyl estradiol | Norethindrone acetate |
| 98 | C31 | Ethinyl estradiol | Norethindrone acetate |
| 99 | C32 | Ethinyl estradiol | Norethindrone acetate |
| 100 | C33 | Ethinyl estradiol | Norethindrone acetate |
| 101 | C34 | Ethinyl estradiol | Norethindrone acetate |
| 102 | C35 | Ethinyl estradiol | Norethindrone acetate |
| 103 | C36 | Ethinyl estradiol | Norethindrone acetate |
| 104 | C37 | Ethinyl estradiol | Norethindrone acetate |
| 105 | C38 | Ethinyl estradiol | Norethindrone acetate |
| 106 | C39 | Ethinyl estradiol | Norethindrone acetate |
| 107 | C40 | Ethinyl estradiol | Norethindrone acetate |
| 108 | C41 | Ethinyl estradiol | Norethindrone acetate |
| 109 | C42 | Ethinyl estradiol | Norethindrone acetate |
| 110 | C43 | Ethinyl estradiol | Norethindrone acetate |
| 111 | C44 | Ethinyl estradiol | Norethindrone acetate |
| 112 | C45 | Ethinyl estradiol | Norethindrone acetate |
| 113 | C46 | Ethinyl estradiol | Norethindrone acetate |
| 114 | C47 | Ethinyl estradiol | Norethindrone acetate |
| 115 | C48 | Ethinyl estradiol | Norethindrone acetate |
| 116 | C49 | Ethinyl estradiol | Norethindrone acetate |
| 117 | C50 | Ethinyl estradiol | Norethindrone acetate |
| 118 | C51 | Ethinyl estradiol | Norethindrone acetate |
| 119 | C52 | Ethinyl estradiol | Norethindrone acetate |
| 120 | C53 | Ethinyl estradiol | Norethindrone acetate |
| 121 | C54 | Ethinyl estradiol | Norethindrone acetate |
| 122 | C55 | Ethinyl estradiol | Norethindrone acetate |
| 123 | C56 | Ethinyl estradiol | Norethindrone acetate |
| 124 | C57 | Ethinyl estradiol | Norethindrone acetate |
| 125 | C58 | Ethinyl estradiol | Norethindrone acetate |
| 126 | C59 | Ethinyl estradiol | Norethindrone acetate |
| 127 | C60 | Ethinyl estradiol | Norethindrone acetate |
| 128 | C61 | Ethinyl estradiol | Norethindrone acetate |
| 129 | C62 | Ethinyl estradiol | Norethindrone acetate |
| 130 | C63 | Ethinyl estradiol | Norethindrone acetate |
| 131 | C64 | Ethinyl estradiol | Norethindrone acetate |
| 132 | C65 | Ethinyl estradiol | Norethindrone acetate |
| 133 | C66 | Ethinyl estradiol | Norethindrone acetate |
| 134 | C67 | Ethinyl estradiol | Norethindrone acetate |
| 135 | C1 | Ethinyl estradiol | Norgestimate |
| 136 | C2 | Ethinyl estradiol | Norgestimate |
| 137 | C3 | Ethinyl estradiol | Norgestimate |
| 138 | C4 | Ethinyl estradiol | Norgestimate |
| 139 | C5 | Ethinyl estradiol | Norgestimate |
| 140 | C6 | Ethinyl estradiol | Norgestimate |
| 141 | C7 | Ethinyl estradiol | Norgestimate |
| 142 | C8 | Ethinyl estradiol | Norgestimate |
| 143 | C9 | Ethinyl estradiol | Norgestimate |
| 144 | C10 | Ethinyl estradiol | Norgestimate |
| 145 | C11 | Ethinyl estradiol | Norgestimate |
| 146 | C12 | Ethinyl estradiol | Norgestimate |
| 147 | C13 | Ethinyl estradiol | Norgestimate |
| 148 | C14 | Ethinyl estradiol | Norgestimate |
| 149 | C15 | Ethinyl estradiol | Norgestimate |
| 150 | C16 | Ethinyl estradiol | Norgestimate |
| 151 | C17 | Ethinyl estradiol | Norgestimate |
| 152 | C18 | Ethinyl estradiol | Norgestimate |
| 153 | C19 | Ethinyl estradiol | Norgestimate |
| 154 | C20 | Ethinyl estradiol | Norgestimate |
| 155 | C21 | Ethinyl estradiol | Norgestimate |
| 156 | C22 | Ethinyl estradiol | Norgestimate |
| 157 | C23 | Ethinyl estradiol | Norgestimate |
| 158 | C24 | Ethinyl estradiol | Norgestimate |
| 159 | C25 | Ethinyl estradiol | Norgestimate |
| 160 | C26 | Ethinyl estradiol | Norgestimate |
| 161 | C27 | Ethinyl estradiol | Norgestimate |
| 162 | C28 | Ethinyl estradiol | Norgestimate |
| 163 | C29 | Ethinyl estradiol | Norgestimate |
| 164 | C30 | Ethinyl estradiol | Norgestimate |
| 165 | C31 | Ethinyl estradiol | Norgestimate |
| 166 | C32 | Ethinyl estradiol | Norgestimate |
| 167 | C33 | Ethinyl estradiol | Norgestimate |
| 168 | C34 | Ethinyl estradiol | Norgestimate |
| 169 | C35 | Ethinyl estradiol | Norgestimate |
| 170 | C36 | Ethinyl estradiol | Norgestimate |
| 171 | C37 | Ethinyl estradiol | Norgestimate |
| 172 | C38 | Ethinyl estradiol | Norgestimate |
| 173 | C39 | Ethinyl estradiol | Norgestimate |
| 174 | C40 | Ethinyl estradiol | Norgestimate |
| 175 | C41 | Ethinyl estradiol | Norgestimate |
| 176 | C42 | Ethinyl estradiol | Norgestimate |
| 177 | C43 | Ethinyl estradiol | Norgestimate |
| 178 | C44 | Ethinyl estradiol | Norgestimate |
| 179 | C45 | Ethinyl estradiol | Norgestimate |
| 180 | C46 | Ethinyl estradiol | Norgestimate |
| 181 | C47 | Ethinyl estradiol | Norgestimate |
| 182 | C48 | Ethinyl estradiol | Norgestimate |
| 183 | C49 | Ethinyl estradiol | Norgestimate |
| 184 | C50 | Ethinyl estradiol | Norgestimate |
| 185 | C51 | Ethinyl estradiol | Norgestimate |
| 186 | C52 | Ethinyl estradiol | Norgestimate |
| 187 | C53 | Ethinyl estradiol | Norgestimate |
| 188 | C54 | Ethinyl estradiol | Norgestimate |
| 189 | C55 | Ethinyl estradiol | Norgestimate |
| 190 | C56 | Ethinyl estradiol | Norgestimate |
| 191 | C57 | Ethinyl estradiol | Norgestimate |
| 192 | C58 | Ethinyl estradiol | Norgestimate |
| 193 | C59 | Ethinyl estradiol | Norgestimate |
| 194 | C60 | Ethinyl estradiol | Norgestimate |
| 195 | C61 | Ethinyl estradiol | Norgestimate |
| 196 | C62 | Ethinyl estradiol | Norgestimate |
| 197 | C63 | Ethinyl estradiol | Norgestimate |
| 198 | C64 | Ethinyl estradiol | Norgestimate |
| 199 | C65 | Ethinyl estradiol | Norgestimate |
| 200 | C66 | Ethinyl estradiol | Norgestimate |
| 201 | C67 | Ethinyl estradiol | Norgestimate |
| 202 | C1 | Ethinyl estradiol | Ethynodiol diacetate |
| 203 | C2 | Ethinyl estradiol | Ethynodiol diacetate |
| 204 | C3 | Ethinyl estradiol | Ethynodiol diacetate |
| 205 | C4 | Ethinyl estradiol | Ethynodiol diacetate |
| 206 | C5 | Ethinyl estradiol | Ethynodiol diacetate |
| 207 | C6 | Ethinyl estradiol | Ethynodiol diacetate |
| 208 | C7 | Ethinyl estradiol | Ethynodiol diacetate |
| 209 | C8 | Ethinyl estradiol | Ethynodiol diacetate |
| 210 | C9 | Ethinyl estradiol | Ethynodiol diacetate |
| 211 | C10 | Ethinyl estradiol | Ethynodiol diacetate |
| 212 | C11 | Ethinyl estradiol | Ethynodiol diacetate |
| 213 | C12 | Ethinyl estradiol | Ethynodiol diacetate |
| 214 | C13 | Ethinyl estradiol | Ethynodiol diacetate |
| 215 | C14 | Ethinyl estradiol | Ethynodiol diacetate |
| 216 | C15 | Ethinyl estradiol | Ethynodiol diacetate |
| 217 | C16 | Ethinyl estradiol | Ethynodiol diacetate |
| 218 | C17 | Ethinyl estradiol | Ethynodiol diacetate |
| 219 | C18 | Ethinyl estradiol | Ethynodiol diacetate |
| 220 | C19 | Ethinyl estradiol | Ethynodiol diacetate |
| 221 | C20 | Ethinyl estradiol | Ethynodiol diacetate |
| 222 | C21 | Ethinyl estradiol | Ethynodiol diacetate |
| 223 | C22 | Ethinyl estradiol | Ethynodiol diacetate |
| 224 | C23 | Ethinyl estradiol | Ethynodiol diacetate |
| 225 | C24 | Ethinyl estradiol | Ethynodiol diacetate |
| 226 | C25 | Ethinyl estradiol | Ethynodiol diacetate |
| 227 | C26 | Ethinyl estradiol | Ethynodiol diacetate |
| 228 | C27 | Ethinyl estradiol | Ethynodiol diacetate |
| 229 | C28 | Ethinyl estradiol | Ethynodiol diacetate |
| 230 | C29 | Ethinyl estradiol | Ethynodiol diacetate |
| 231 | C30 | Ethinyl estradiol | Ethynodiol diacetate |
| 232 | C31 | Ethinyl estradiol | Ethynodiol diacetate |
| 233 | C32 | Ethinyl estradiol | Ethynodiol diacetate |
| 234 | C33 | Ethinyl estradiol | Ethynodiol diacetate |
| 235 | C34 | Ethinyl estradiol | Ethynodiol diacetate |
| 236 | C35 | Ethinyl estradiol | Ethynodiol diacetate |
| 237 | C36 | Ethinyl estradiol | Ethynodiol diacetate |
| 238 | C37 | Ethinyl estradiol | Ethynodiol diacetate |
| 239 | C38 | Ethinyl estradiol | Ethynodiol diacetate |
| 240 | C39 | Ethinyl estradiol | Ethynodiol diacetate |
| 241 | C40 | Ethinyl estradiol | Ethynodiol diacetate |
| 242 | C41 | Ethinyl estradiol | Ethynodiol diacetate |
| 243 | C42 | Ethinyl estradiol | Ethynodiol diacetate |
| 244 | C43 | Ethinyl estradiol | Ethynodiol diacetate |
| 245 | C44 | Ethinyl estradiol | Ethynodiol diacetate |
| 246 | C45 | Ethinyl estradiol | Ethynodiol diacetate |
| 247 | C46 | Ethinyl estradiol | Ethynodiol diacetate |
| 248 | C47 | Ethinyl estradiol | Ethynodiol diacetate |
| 249 | C48 | Ethinyl estradiol | Ethynodiol diacetate |
| 250 | C49 | Ethinyl estradiol | Ethynodiol diacetate |
| 251 | C50 | Ethinyl estradiol | Ethynodiol diacetate |
| 252 | C51 | Ethinyl estradiol | Ethynodiol diacetate |
| 253 | C52 | Ethinyl estradiol | Ethynodiol diacetate |
| 254 | C53 | Ethinyl estradiol | Ethynodiol diacetate |
| 255 | C54 | Ethinyl estradiol | Ethynodiol diacetate |
| 256 | C55 | Ethinyl estradiol | Ethynodiol diacetate |
| 257 | C56 | Ethinyl estradiol | Ethynodiol diacetate |
| 258 | C57 | Ethinyl estradiol | Ethynodiol diacetate |
| 259 | C58 | Ethinyl estradiol | Ethynodiol diacetate |
| 260 | C59 | Ethinyl estradiol | Ethynodiol diacetate |
| 261 | C60 | Ethinyl estradiol | Ethynodiol diacetate |
| 262 | C61 | Ethinyl estradiol | Ethynodiol diacetate |
| 263 | C62 | Ethinyl estradiol | Ethynodiol diacetate |
| 264 | C63 | Ethinyl estradiol | Ethynodiol diacetate |
| 265 | C64 | Ethinyl estradiol | Ethynodiol diacetate |
| 266 | C65 | Ethinyl estradiol | Ethynodiol diacetate |
| 267 | C66 | Ethinyl estradiol | Ethynodiol diacetate |
| 268 | C67 | Ethinyl estradiol | Ethynodiol diacetate |
| 269 | C1 | Ethinyl estradiol | Desogestrel |
| 270 | C2 | Ethinyl estradiol | Desogestrel |
| 271 | C3 | Ethinyl estradiol | Desogestrel |
| 272 | C4 | Ethinyl estradiol | Desogestrel |
| 273 | C5 | Ethinyl estradiol | Desogestrel |
| 274 | C6 | Ethinyl estradiol | Desogestrel |
| 275 | C7 | Ethinyl estradiol | Desogestrel |
| 276 | C8 | Ethinyl estradiol | Desogestrel |
| 277 | C9 | Ethinyl estradiol | Desogestrel |
| 278 | C10 | Ethinyl estradiol | Desogestrel |
| 279 | C11 | Ethinyl estradiol | Desogestrel |
| 280 | C12 | Ethinyl estradiol | Desogestrel |
| 281 | C13 | Ethinyl estradiol | Desogestrel |
| 282 | C14 | Ethinyl estradiol | Desogestrel |
| 283 | C15 | Ethinyl estradiol | Desogestrel |
| 284 | C16 | Ethinyl estradiol | Desogestrel |
| 285 | C17 | Ethinyl estradiol | Desogestrel |
| 286 | C18 | Ethinyl estradiol | Desogestrel |
| 287 | C19 | Ethinyl estradiol | Desogestrel |
| 288 | C20 | Ethinyl estradiol | Desogestrel |
| 289 | C21 | Ethinyl estradiol | Desogestrel |
| 290 | C22 | Ethinyl estradiol | Desogestrel |
| 291 | C23 | Ethinyl estradiol | Desogestrel |
| 292 | C24 | Ethinyl estradiol | Desogestrel |
| 293 | C25 | Ethinyl estradiol | Desogestrel |
| 294 | C26 | Ethinyl estradiol | Desogestrel |
| 295 | C27 | Ethinyl estradiol | Desogestrel |
| 296 | C28 | Ethinyl estradiol | Desogestrel |
| 297 | C29 | Ethinyl estradiol | Desogestrel |
| 298 | C30 | Ethinyl estradiol | Desogestrel |
| 299 | C31 | Ethinyl estradiol | Desogestrel |
| 300 | C32 | Ethinyl estradiol | Desogestrel |
| 301 | C33 | Ethinyl estradiol | Desogestrel |
| 302 | C34 | Ethinyl estradiol | Desogestrel |
| 303 | C35 | Ethinyl estradiol | Desogestrel |
| 304 | C36 | Ethinyl estradiol | Desogestrel |
| 305 | C37 | Ethinyl estradiol | Desogestrel |
| 306 | C38 | Ethinyl estradiol | Desogestrel |
| 307 | C39 | Ethinyl estradiol | Desogestrel |
| 308 | C40 | Ethinyl estradiol | Desogestrel |
| 309 | C41 | Ethinyl estradiol | Desogestrel |
| 310 | C42 | Ethinyl estradiol | Desogestrel |
| 311 | C43 | Ethinyl estradiol | Desogestrel |
| 312 | C44 | Ethinyl estradiol | Desogestrel |
| 313 | C45 | Ethinyl estradiol | Desogestrel |
| 314 | C46 | Ethinyl estradiol | Desogestrel |
| 315 | C47 | Ethinyl estradiol | Desogestrel |
| 316 | C48 | Ethinyl estradiol | Desogestrel |
| 317 | C49 | Ethinyl estradiol | Desogestrel |
| 318 | C50 | Ethinyl estradiol | Desogestrel |
| 319 | C51 | Ethinyl estradiol | Desogestrel |
| 320 | C52 | Ethinyl estradiol | Desogestrel |
| 321 | C53 | Ethinyl estradiol | Desogestrel |
| 322 | C54 | Ethinyl estradiol | Desogestrel |
| 323 | C55 | Ethinyl estradiol | Desogestrel |
| 324 | C56 | Ethinyl estradiol | Desogestrel |
| 325 | C57 | Ethinyl estradiol | Desogestrel |
| 326 | C58 | Ethinyl estradiol | Desogestrel |
| 327 | C59 | Ethinyl estradiol | Desogestrel |
| 328 | C60 | Ethinyl estradiol | Desogestrel |
| 329 | C61 | Ethinyl estradiol | Desogestrel |
| 330 | C62 | Ethinyl estradiol | Desogestrel |
| 331 | C63 | Ethinyl estradiol | Desogestrel |
| 332 | C64 | Ethinyl estradiol | Desogestrel |
| 333 | C65 | Ethinyl estradiol | Desogestrel |
| 334 | C66 | Ethinyl estradiol | Desogestrel |
| 335 | C67 | Ethinyl estradiol | Desogestrel |
| 336 | C1 | Ethinyl estradiol | Norgestrel |
| 337 | C2 | Ethinyl estradiol | Norgestrel |
| 338 | C3 | Ethinyl estradiol | Norgestrel |
| 339 | C4 | Ethinyl estradiol | Norgestrel |
| 340 | C5 | Ethinyl estradiol | Norgestrel |
| 341 | C6 | Ethinyl estradiol | Norgestrel |
| 342 | C7 | Ethinyl estradiol | Norgestrel |
| 343 | C8 | Ethinyl estradiol | Norgestrel |
| 344 | C9 | Ethinyl estradiol | Norgestrel |
| 345 | C10 | Ethinyl estradiol | Norgestrel |
| 346 | C11 | Ethinyl estradiol | Norgestrel |
| 347 | C12 | Ethinyl estradiol | Norgestrel |
| 348 | C13 | Ethinyl estradiol | Norgestrel |
| 349 | C14 | Ethinyl estradiol | Norgestrel |
| 350 | C15 | Ethinyl estradiol | Norgestrel |
| 351 | C16 | Ethinyl estradiol | Norgestrel |
| 352 | C17 | Ethinyl estradiol | Norgestrel |
| 353 | C18 | Ethinyl estradiol | Norgestrel |
| 354 | C19 | Ethinyl estradiol | Norgestrel |
| 355 | C20 | Ethinyl estradiol | Norgestrel |
| 356 | C21 | Ethinyl estradiol | Norgestrel |
| 357 | C22 | Ethinyl estradiol | Norgestrel |
| 358 | C23 | Ethinyl estradiol | Norgestrel |
| 359 | C24 | Ethinyl estradiol | Norgestrel |
| 360 | C25 | Ethinyl estradiol | Norgestrel |
| 361 | C26 | Ethinyl estradiol | Norgestrel |
| 362 | C27 | Ethinyl estradiol | Norgestrel |
| 363 | C28 | Ethinyl estradiol | Norgestrel |
| 364 | C29 | Ethinyl estradiol | Norgestrel |
| 365 | C30 | Ethinyl estradiol | Norgestrel |
| 366 | C31 | Ethinyl estradiol | Norgestrel |
| 367 | C32 | Ethinyl estradiol | Norgestrel |
| 368 | C33 | Ethinyl estradiol | Norgestrel |
| 369 | C34 | Ethinyl estradiol | Norgestrel |
| 370 | C35 | Ethinyl estradiol | Norgestrel |
| 371 | C36 | Ethinyl estradiol | Norgestrel |
| 372 | C37 | Ethinyl estradiol | Norgestrel |
| 373 | C38 | Ethinyl estradiol | Norgestrel |
| 374 | C39 | Ethinyl estradiol | Norgestrel |
| 375 | C40 | Ethinyl estradiol | Norgestrel |
| 376 | C41 | Ethinyl estradiol | Norgestrel |
| 377 | C42 | Ethinyl estradiol | Norgestrel |
| 378 | C43 | Ethinyl estradiol | Norgestrel |
| 379 | C44 | Ethinyl estradiol | Norgestrel |
| 380 | C45 | Ethinyl estradiol | Norgestrel |
| 381 | C46 | Ethinyl estradiol | Norgestrel |
| 382 | C47 | Ethinyl estradiol | Norgestrel |
| 383 | C48 | Ethinyl estradiol | Norgestrel |
| 384 | C49 | Ethinyl estradiol | Norgestrel |
| 385 | C50 | Ethinyl estradiol | Norgestrel |
| 386 | C51 | Ethinyl estradiol | Norgestrel |
| 387 | C52 | Ethinyl estradiol | Norgestrel |
| 388 | C53 | Ethinyl estradiol | Norgestrel |
| 389 | C54 | Ethinyl estradiol | Norgestrel |
| 390 | C55 | Ethinyl estradiol | Norgestrel |
| 391 | C56 | Ethinyl estradiol | Norgestrel |
| 392 | C57 | Ethinyl estradiol | Norgestrel |
| 393 | C58 | Ethinyl estradiol | Norgestrel |
| 394 | C59 | Ethinyl estradiol | Norgestrel |
| 395 | C60 | Ethinyl estradiol | Norgestrel |
| 396 | C61 | Ethinyl estradiol | Norgestrel |
| 397 | C62 | Ethinyl estradiol | Norgestrel |
| 398 | C63 | Ethinyl estradiol | Norgestrel |
| 399 | C64 | Ethinyl estradiol | Norgestrel |
| 400 | C65 | Ethinyl estradiol | Norgestrel |
| 401 | C66 | Ethinyl estradiol | Norgestrel |
| 402 | C67 | Ethinyl estradiol | Norgestrel |
| 403 | C1 | Ethinyl estradiol | Norethindrone |
| 404 | C2 | Ethinyl estradiol | Norethindrone |
| 405 | C3 | Ethinyl estradiol | Norethindrone |
| 406 | C4 | Ethinyl estradiol | Norethindrone |
| 407 | C5 | Ethinyl estradiol | Norethindrone |
| 408 | C6 | Ethinyl estradiol | Norethindrone |
| 409 | C7 | Ethinyl estradiol | Norethindrone |
| 410 | C8 | Ethinyl estradiol | Norethindrone |
| 411 | C9 | Ethinyl estradiol | Norethindrone |
| 412 | C10 | Ethinyl estradiol | Norethindrone |
| 413 | C11 | Ethinyl estradiol | Norethindrone |
| 414 | C12 | Ethinyl estradiol | Norethindrone |
| 415 | C13 | Ethinyl estradiol | Norethindrone |
| 416 | C14 | Ethinyl estradiol | Norethindrone |
| 417 | C15 | Ethinyl estradiol | Norethindrone |
| 418 | C16 | Ethinyl estradiol | Norethindrone |
| 419 | C17 | Ethinyl estradiol | Norethindrone |
| 420 | C18 | Ethinyl estradiol | Norethindrone |
| 421 | C19 | Ethinyl estradiol | Norethindrone |
| 422 | C20 | Ethinyl estradiol | Norethindrone |
| 423 | C21 | Ethinyl estradiol | Norethindrone |
| 424 | C22 | Ethinyl estradiol | Norethindrone |
| 425 | C23 | Ethinyl estradiol | Norethindrone |
| 426 | C24 | Ethinyl estradiol | Norethindrone |
| 427 | C25 | Ethinyl estradiol | Norethindrone |
| 428 | C26 | Ethinyl estradiol | Norethindrone |
| 429 | C27 | Ethinyl estradiol | Norethindrone |
| 430 | C28 | Ethinyl estradiol | Norethindrone |
| 431 | C29 | Ethinyl estradiol | Norethindrone |
| 432 | C30 | Ethinyl estradiol | Norethindrone |
| 433 | C31 | Ethinyl estradiol | Norethindrone |
| 434 | C32 | Ethinyl estradiol | Norethindrone |
| 435 | C33 | Ethinyl estradiol | Norethindrone |
| 436 | C34 | Ethinyl estradiol | Norethindrone |
| 437 | C35 | Ethinyl estradiol | Norethindrone |
| 438 | C36 | Ethinyl estradiol | Norethindrone |
| 439 | C37 | Ethinyl estradiol | Norethindrone |
| 440 | C38 | Ethinyl estradiol | Norethindrone |
| 441 | C39 | Ethinyl estradiol | Norethindrone |
| 442 | C40 | Ethinyl estradiol | Norethindrone |
| 443 | C41 | Ethinyl estradiol | Norethindrone |
| 444 | C42 | Ethinyl estradiol | Norethindrone |
| 445 | C43 | Ethinyl estradiol | Norethindrone |
| 446 | C44 | Ethinyl estradiol | Norethindrone |
| 447 | C45 | Ethinyl estradiol | Norethindrone |
| 448 | C46 | Ethinyl estradiol | Norethindrone |
| 449 | C47 | Ethinyl estradiol | Norethindrone |
| 450 | C48 | Ethinyl estradiol | Norethindrone |
| 451 | C49 | Ethinyl estradiol | Norethindrone |
| 452 | C50 | Ethinyl estradiol | Norethindrone |
| 453 | C51 | Ethinyl estradiol | Norethindrone |
| 454 | C52 | Ethinyl estradiol | Norethindrone |
| 455 | C53 | Ethinyl estradiol | Norethindrone |
| 456 | C54 | Ethinyl estradiol | Norethindrone |
| 457 | C55 | Ethinyl estradiol | Norethindrone |
| 458 | C56 | Ethinyl estradiol | Norethindrone |
| 459 | C57 | Ethinyl estradiol | Norethindrone |
| 460 | C58 | Ethinyl estradiol | Norethindrone |
| 461 | C59 | Ethinyl estradiol | Norethindrone |
| 462 | C60 | Ethinyl estradiol | Norethindrone |
| 463 | C61 | Ethinyl estradiol | Norethindrone |
| 464 | C62 | Ethinyl estradiol | Norethindrone |
| 465 | C63 | Ethinyl estradiol | Norethindrone |
| 466 | C64 | Ethinyl estradiol | Norethindrone |
| 467 | C65 | Ethinyl estradiol | Norethindrone |
| 468 | C66 | Ethinyl estradiol | Norethindrone |
| 469 | C67 | Ethinyl estradiol | Norethindrone |
| 470 | C1 | Ethinyl estradiol | 3-Ketodesogestrel |
| 471 | C2 | Ethinyl estradiol | 3-Ketodesogestrel |
| 472 | C3 | Ethinyl estradiol | 3-Ketodesogestrel |
| 473 | C4 | Ethinyl estradiol | 3-Ketodesogestrel |
| 474 | C5 | Ethinyl estradiol | 3-Ketodesogestrel |
| 475 | C6 | Ethinyl estradiol | 3-Ketodesogestrel |
| 476 | C7 | Ethinyl estradiol | 3-Ketodesogestrel |
| 477 | C8 | Ethinyl estradiol | 3-Ketodesogestrel |
| 478 | C9 | Ethinyl estradiol | 3-Ketodesogestrel |
| 479 | C10 | Ethinyl estradiol | 3-Ketodesogestrel |
| 480 | C11 | Ethinyl estradiol | 3-Ketodesogestrel |
| 481 | C12 | Ethinyl estradiol | 3-Ketodesogestrel |
| 482 | C13 | Ethinyl estradiol | 3-Ketodesogestrel |
| 483 | C14 | Ethinyl estradiol | 3-Ketodesogestrel |
| 484 | C15 | Ethinyl estradiol | 3-Ketodesogestrel |
| 485 | C16 | Ethinyl estradiol | 3-Ketodesogestrel |
| 486 | C17 | Ethinyl estradiol | 3-Ketodesogestrel |
| 487 | C18 | Ethinyl estradiol | 3-Ketodesogestrel |
| 488 | C19 | Ethinyl estradiol | 3-Ketodesogestrel |
| 489 | C20 | Ethinyl estradiol | 3-Ketodesogestrel |
| 490 | C21 | Ethinyl estradiol | 3-Ketodesogestrel |
| 491 | C22 | Ethinyl estradiol | 3-Ketodesogestrel |
| 492 | C23 | Ethinyl estradiol | 3-Ketodesogestrel |
| 493 | C24 | Ethinyl estradiol | 3-Ketodesogestrel |
| 494 | C25 | Ethinyl estradiol | 3-Ketodesogestrel |
| 495 | C26 | Ethinyl estradiol | 3-Ketodesogestrel |
| 496 | C27 | Ethinyl estradiol | 3-Ketodesogestrel |
| 497 | C28 | Ethinyl estradiol | 3-Ketodesogestrel |
| 498 | C29 | Ethinyl estradiol | 3-Ketodesogestrel |
| 499 | C30 | Ethinyl estradiol | 3-Ketodesogestrel |
| 500 | C31 | Ethinyl estradiol | 3-Ketodesogestrel |
| 501 | C32 | Ethinyl estradiol | 3-Ketodesogestrel |
| 502 | C33 | Ethinyl estradiol | 3-Ketodesogestrel |
| 503 | C34 | Ethinyl estradiol | 3-Ketodesogestrel |
| 504 | C35 | Ethinyl estradiol | 3-Ketodesogestrel |
| 505 | C36 | Ethinyl estradiol | 3-Ketodesogestrel |
| 506 | C37 | Ethinyl estradiol | 3-Ketodesogestrel |
| 507 | C38 | Ethinyl estradiol | 3-Ketodesogestrel |
| 508 | C39 | Ethinyl estradiol | 3-Ketodesogestrel |
| 509 | C40 | Ethinyl estradiol | 3-Ketodesogestrel |
| 510 | C41 | Ethinyl estradiol | 3-Ketodesogestrel |
| 511 | C42 | Ethinyl estradiol | 3-Ketodesogestrel |
| 512 | C43 | Ethinyl estradiol | 3-Ketodesogestrel |
| 513 | C44 | Ethinyl estradiol | 3-Ketodesogestrel |
| 514 | C45 | Ethinyl estradiol | 3-Ketodesogestrel |
| 515 | C46 | Ethinyl estradiol | 3-Ketodesogestrel |
| 516 | C47 | Ethinyl estradiol | 3-Ketodesogestrel |
| 517 | C48 | Ethinyl estradiol | 3-Ketodesogestrel |
| 518 | C49 | Ethinyl estradiol | 3-Ketodesogestrel |
| 519 | C50 | Ethinyl estradiol | 3-Ketodesogestrel |
| 520 | C51 | Ethinyl estradiol | 3-Ketodesogestrel |
| 521 | C52 | Ethinyl estradiol | 3-Ketodesogestrel |
| 522 | C53 | Ethinyl estradiol | 3-Ketodesogestrel |
| 523 | C54 | Ethinyl estradiol | 3-Ketodesogestrel |
| 524 | C55 | Ethinyl estradiol | 3-Ketodesogestrel |
| 525 | C56 | Ethinyl estradiol | 3-Ketodesogestrel |
| 526 | C57 | Ethinyl estradiol | 3-Ketodesogestrel |
| 527 | C58 | Ethinyl estradiol | 3-Ketodesogestrel |
| 528 | C59 | Ethinyl estradiol | 3-Ketodesogestrel |
| 529 | C60 | Ethinyl estradiol | 3-Ketodesogestrel |
| 530 | C61 | Ethinyl estradiol | 3-Ketodesogestrel |
| 531 | C62 | Ethinyl estradiol | 3-Ketodesogestrel |
| 532 | C63 | Ethinyl estradiol | 3-Ketodesogestrel |
| 533 | C64 | Ethinyl estradiol | 3-Ketodesogestrel |
| 534 | C65 | Ethinyl estradiol | 3-Ketodesogestrel |
| 535 | C66 | Ethinyl estradiol | 3-Ketodesogestrel |
| 536 | C67 | Ethinyl estradiol | 3-Ketodesogestrel |
| 537 | C1 | Ethinyl estradiol | Gestodene |
| 538 | C2 | Ethinyl estradiol | Gestodene |
| 539 | C3 | Ethinyl estradiol | Gestodene |
| 540 | C4 | Ethinyl estradiol | Gestodene |
| 541 | C5 | Ethinyl estradiol | Gestodene |
| 542 | C6 | Ethinyl estradiol | Gestodene |
| 543 | C7 | Ethinyl estradiol | Gestodene |
| 544 | C8 | Ethinyl estradiol | Gestodene |
| 545 | C9 | Ethinyl estradiol | Gestodene |
| 546 | C10 | Ethinyl estradiol | Gestodene |
| 547 | C11 | Ethinyl estradiol | Gestodene |
| 548 | C12 | Ethinyl estradiol | Gestodene |
| 549 | C13 | Ethinyl estradiol | Gestodene |
| 550 | C14 | Ethinyl estradiol | Gestodene |
| 551 | C15 | Ethinyl estradiol | Gestodene |
| 552 | C16 | Ethinyl estradiol | Gestodene |
| 553 | C17 | Ethinyl estradiol | Gestodene |
| 554 | C18 | Ethinyl estradiol | Gestodene |
| 555 | C19 | Ethinyl estradiol | Gestodene |
| 556 | C20 | Ethinyl estradiol | Gestodene |
| 557 | C21 | Ethinyl estradiol | Gestodene |
| 558 | C22 | Ethinyl estradiol | Gestodene |
| 559 | C23 | Ethinyl estradiol | Gestodene |
| 560 | C24 | Ethinyl estradiol | Gestodene |
| 561 | C25 | Ethinyl estradiol | Gestodene |
| 562 | C26 | Ethinyl estradiol | Gestodene |
| 563 | C27 | Ethinyl estradiol | Gestodene |
| 564 | C28 | Ethinyl estradiol | Gestodene |
| 565 | C29 | Ethinyl estradiol | Gestodene |
| 566 | C30 | Ethinyl estradiol | Gestodene |
| 567 | C31 | Ethinyl estradiol | Gestodene |
| 568 | C32 | Ethinyl estradiol | Gestodene |
| 569 | C33 | Ethinyl estradiol | Gestodene |
| 570 | C34 | Ethinyl estradiol | Gestodene |
| 571 | C35 | Ethinyl estradiol | Gestodene |
| 572 | C36 | Ethinyl estradiol | Gestodene |
| 573 | C37 | Ethinyl estradiol | Gestodene |
| 574 | C38 | Ethinyl estradiol | Gestodene |
| 575 | C39 | Ethinyl estradiol | Gestodene |
| 576 | C40 | Ethinyl estradiol | Gestodene |
| 577 | C41 | Ethinyl estradiol | Gestodene |
| 578 | C42 | Ethinyl estradiol | Gestodene |
| 579 | C43 | Ethinyl estradiol | Gestodene |
| 580 | C44 | Ethinyl estradiol | Gestodene |
| 581 | C45 | Ethinyl estradiol | Gestodene |
| 582 | C46 | Ethinyl estradiol | Gestodene |
| 583 | C47 | Ethinyl estradiol | Gestodene |
| 584 | C48 | Ethinyl estradiol | Gestodene |
| 585 | C49 | Ethinyl estradiol | Gestodene |
| 586 | C50 | Ethinyl estradiol | Gestodene |
| 587 | C51 | Ethinyl estradiol | Gestodene |
| 588 | C52 | Ethinyl estradiol | Gestodene |
| 589 | C53 | Ethinyl estradiol | Gestodene |
| 590 | C54 | Ethinyl estradiol | Gestodene |
| 591 | C55 | Ethinyl estradiol | Gestodene |
| 592 | C56 | Ethinyl estradiol | Gestodene |
| 593 | C57 | Ethinyl estradiol | Gestodene |
| 594 | C58 | Ethinyl estradiol | Gestodene |
| 595 | C59 | Ethinyl estradiol | Gestodene |
| 596 | C60 | Ethinyl estradiol | Gestodene |
| 597 | C61 | Ethinyl estradiol | Gestodene |
| 598 | C62 | Ethinyl estradiol | Gestodene |
| 599 | C63 | Ethinyl estradiol | Gestodene |
| 600 | C64 | Ethinyl estradiol | Gestodene |
| 601 | C65 | Ethinyl estradiol | Gestodene |
| 602 | C66 | Ethinyl estradiol | Gestodene |
| 603 | C67 | Ethinyl estradiol | Gestodene |
| 604 | C1 | Ethinyl estradiol | Org 30659 |
| 605 | C2 | Ethinyl estradiol | Org 30659 |
| 606 | C3 | Ethinyl estradiol | Org 30659 |
| 607 | C4 | Ethinyl estradiol | Org 30659 |
| 608 | C5 | Ethinyl estradiol | Org 30659 |
| 609 | C6 | Ethinyl estradiol | Org 30659 |
| 610 | C7 | Ethinyl estradiol | Org 30659 |
| 611 | C8 | Ethinyl estradiol | Org 30659 |
| 612 | C9 | Ethinyl estradiol | Org 30659 |
| 613 | C10 | Ethinyl estradiol | Org 30659 |
| 614 | C11 | Ethinyl estradiol | Org 30659 |
| 615 | C12 | Ethinyl estradiol | Org 30659 |
| 616 | C13 | Ethinyl estradiol | Org 30659 |
| 617 | C14 | Ethinyl estradiol | Org 30659 |
| 618 | C15 | Ethinyl estradiol | Org 30659 |
| 619 | C16 | Ethinyl estradiol | Org 30659 |
| 620 | C17 | Ethinyl estradiol | Org 30659 |
| 621 | C18 | Ethinyl estradiol | Org 30659 |
| 622 | C19 | Ethinyl estradiol | Org 30659 |
| 623 | C20 | Ethinyl estradiol | Org 30659 |
| 624 | C21 | Ethinyl estradiol | Org 30659 |
| 625 | C22 | Ethinyl estradiol | Org 30659 |
| 626 | C23 | Ethinyl estradiol | Org 30659 |
| 627 | C24 | Ethinyl estradiol | Org 30659 |
| 628 | C25 | Ethinyl estradiol | Org 30659 |
| 629 | C26 | Ethinyl estradiol | Org 30659 |
| 630 | C27 | Ethinyl estradiol | Org 30659 |
| 631 | C28 | Ethinyl estradiol | Org 30659 |
| 632 | C29 | Ethinyl estradiol | Org 30659 |
| 633 | C30 | Ethinyl estradiol | Org 30659 |
| 634 | C31 | Ethinyl estradiol | Org 30659 |
| 635 | C32 | Ethinyl estradiol | Org 30659 |
| 636 | C33 | Ethinyl estradiol | Org 30659 |
| 637 | C34 | Ethinyl estradiol | Org 30659 |
| 638 | C35 | Ethinyl estradiol | Org 30659 |
| 639 | C36 | Ethinyl estradiol | Org 30659 |
| 640 | C37 | Ethinyl estradiol | Org 30659 |
| 641 | C38 | Ethinyl estradiol | Org 30659 |
| 642 | C39 | Ethinyl estradiol | Org 30659 |
| 643 | C40 | Ethinyl estradiol | Org 30659 |
| 644 | C41 | Ethinyl estradiol | Org 30659 |
| 645 | C42 | Ethinyl estradiol | Org 30659 |
| 646 | C43 | Ethinyl estradiol | Org 30659 |
| 647 | C44 | Ethinyl estradiol | Org 30659 |
| 648 | C45 | Ethinyl estradiol | Org 30659 |
| 649 | C46 | Ethinyl estradiol | Org 30659 |
| 650 | C47 | Ethinyl estradiol | Org 30659 |
| 651 | C48 | Ethinyl estradiol | Org 30659 |
| 652 | C49 | Ethinyl estradiol | Org 30659 |
| 653 | C50 | Ethinyl estradiol | Org 30659 |
| 654 | C51 | Ethinyl estradiol | Org 30659 |
| 655 | C52 | Ethinyl estradiol | Org 30659 |
| 656 | C53 | Ethinyl estradiol | Org 30659 |
| 657 | C54 | Ethinyl estradiol | Org 30659 |
| 658 | C55 | Ethinyl estradiol | Org 30659 |
| 659 | C56 | Ethinyl estradiol | Org 30659 |
| 660 | C57 | Ethinyl estradiol | Org 30659 |
| 661 | C58 | Ethinyl estradiol | Org 30659 |
| 662 | C59 | Ethinyl estradiol | Org 30659 |
| 663 | C60 | Ethinyl estradiol | Org 30659 |
| 664 | C61 | Ethinyl estradiol | Org 30659 |
| 665 | C62 | Ethinyl estradiol | Org 30659 |
| 666 | C63 | Ethinyl estradiol | Org 30659 |
| 667 | C64 | Ethinyl estradiol | Org 30659 |
| 668 | C65 | Ethinyl estradiol | Org 30659 |
| 669 | C66 | Ethinyl estradiol | Org 30659 |
| 670 | C67 | Ethinyl estradiol | Org 30659 |
| 671 | C1 | Ethinyl estradiol | Trimegestone |
| 672 | C2 | Ethinyl estradiol | Trimegestone |
| 673 | C3 | Ethinyl estradiol | Trimegestone |
| 674 | C4 | Ethinyl estradiol | Trimegestone |
| 675 | C5 | Ethinyl estradiol | Trimegestone |
| 676 | C6 | Ethinyl estradiol | Trimegestone |
| 677 | C7 | Ethinyl estradiol | Trimegestone |
| 678 | C8 | Ethinyl estradiol | Trimegestone |
| 679 | C9 | Ethinyl estradiol | Trimegestone |
| 680 | C10 | Ethinyl estradiol | Trimegestone |
| 681 | C11 | Ethinyl estradiol | Trimegestone |
| 682 | C12 | Ethinyl estradiol | Trimegestone |
| 683 | C13 | Ethinyl estradiol | Trimegestone |
| 684 | C14 | Ethinyl estradiol | Trimegestone |
| 685 | C15 | Ethinyl estradiol | Trimegestone |
| 686 | C16 | Ethinyl estradiol | Trimegestone |
| 687 | C17 | Ethinyl estradiol | Trimegestone |
| 688 | C18 | Ethinyl estradiol | Trimegestone |
| 689 | C19 | Ethinyl estradiol | Trimegestone |
| 690 | C20 | Ethinyl estradiol | Trimegestone |
| 691 | C21 | Ethinyl estradiol | Trimegestone |
| 692 | C22 | Ethinyl estradiol | Trimegestone |
| 693 | C23 | Ethinyl estradiol | Trimegestone |
| 694 | C24 | Ethinyl estradiol | Trimegestone |
| 695 | C25 | Ethinyl estradiol | Trimegestone |
| 696 | C26 | Ethinyl estradiol | Trimegestone |
| 697 | C27 | Ethinyl estradiol | Trimegestone |
| 698 | C28 | Ethinyl estradiol | Trimegestone |
| 699 | C29 | Ethinyl estradiol | Trimegestone |
| 700 | C30 | Ethinyl estradiol | Trimegestone |
| 701 | C31 | Ethinyl estradiol | Trimegestone |
| 702 | C32 | Ethinyl estradiol | Trimegestone |
| 703 | C33 | Ethinyl estradiol | Trimegestone |
| 704 | C34 | Ethinyl estradiol | Trimegestone |
| 705 | C35 | Ethinyl estradiol | Trimegestone |
| 706 | C36 | Ethinyl estradiol | Trimegestone |
| 707 | C37 | Ethinyl estradiol | Trimegestone |
| 708 | C38 | Ethinyl estradiol | Trimegestone |
| 709 | C39 | Ethinyl estradiol | Trimegestone |
| 710 | C40 | Ethinyl estradiol | Trimegestone |
| 711 | C41 | Ethinyl estradiol | Trimegestone |
| 712 | C42 | Ethinyl estradiol | Trimegestone |
| 713 | C43 | Ethinyl estradiol | Trimegestone |
| 714 | C44 | Ethinyl estradiol | Trimegestone |
| 715 | C45 | Ethinyl estradiol | Trimegestone |
| 716 | C46 | Ethinyl estradiol | Trimegestone |
| 717 | C47 | Ethinyl estradiol | Trimegestone |
| 718 | C48 | Ethinyl estradiol | Trimegestone |
| 719 | C49 | Ethinyl estradiol | Trimegestone |
| 720 | C50 | Ethinyl estradiol | Trimegestone |
| 721 | C51 | Ethinyl estradiol | Trimegestone |
| 722 | C52 | Ethinyl estradiol | Trimegestone |
| 723 | C53 | Ethinyl estradiol | Trimegestone |
| 724 | C54 | Ethinyl estradiol | Trimegestone |
| 725 | C55 | Ethinyl estradiol | Trimegestone |
| 726 | C56 | Ethinyl estradiol | Trimegestone |
| 727 | C57 | Ethinyl estradiol | Trimegestone |
| 728 | C58 | Ethinyl estradiol | Trimegestone |
| 729 | C59 | Ethinyl estradiol | Trimegestone |
| 730 | C60 | Ethinyl estradiol | Trimegestone |
| 731 | C61 | Ethinyl estradiol | Trimegestone |
| 732 | C62 | Ethinyl estradiol | Trimegestone |
| 733 | C63 | Ethinyl estradiol | Trimegestone |
| 734 | C64 | Ethinyl estradiol | Trimegestone |
| 735 | C65 | Ethinyl estradiol | Trimegestone |
| 736 | C66 | Ethinyl estradiol | Trimegestone |
| 737 | C67 | Ethinyl estradiol | Trimegestone |
| 738 | C1 | Ethinyl estradiol | Dienogest |
| 739 | C2 | Ethinyl estradiol | Dienogest |
| 740 | C3 | Ethinyl estradiol | Dienogest |
| 741 | C4 | Ethinyl estradiol | Dienogest |
| 742 | C5 | Ethinyl estradiol | Dienogest |
| 743 | C6 | Ethinyl estradiol | Dienogest |
| 744 | C7 | Ethinyl estradiol | Dienogest |
| 745 | C8 | Ethinyl estradiol | Dienogest |
| 746 | C9 | Ethinyl estradiol | Dienogest |
| 747 | C10 | Ethinyl estradiol | Dienogest |
| 748 | C11 | Ethinyl estradiol | Dienogest |
| 749 | C12 | Ethinyl estradiol | Dienogest |
| 750 | C13 | Ethinyl estradiol | Dienogest |
| 751 | C14 | Ethinyl estradiol | Dienogest |
| 752 | C15 | Ethinyl estradiol | Dienogest |
| 753 | C16 | Ethinyl estradiol | Dienogest |
| 754 | C17 | Ethinyl estradiol | Dienogest |
| 755 | C18 | Ethinyl estradiol | Dienogest |
| 756 | C19 | Ethinyl estradiol | Dienogest |
| 757 | C20 | Ethinyl estradiol | Dienogest |
| 758 | C21 | Ethinyl estradiol | Dienogest |
| 759 | C22 | Ethinyl estradiol | Dienogest |
| 760 | C23 | Ethinyl estradiol | Dienogest |
| 761 | C24 | Ethinyl estradiol | Dienogest |
| 762 | C25 | Ethinyl estradiol | Dienogest |
| 763 | C26 | Ethinyl estradiol | Dienogest |
| 764 | C27 | Ethinyl estradiol | Dienogest |
| 765 | C28 | Ethinyl estradiol | Dienogest |
| 766 | C29 | Ethinyl estradiol | Dienogest |
| 767 | C30 | Ethinyl estradiol | Dienogest |
| 768 | C31 | Ethinyl estradiol | Dienogest |
| 769 | C32 | Ethinyl estradiol | Dienogest |
| 770 | C33 | Ethinyl estradiol | Dienogest |
| 771 | C34 | Ethinyl estradiol | Dienogest |
| 772 | C35 | Ethinyl estradiol | Dienogest |
| 773 | C36 | Ethinyl estradiol | Dienogest |
| 774 | C37 | Ethinyl estradiol | Dienogest |
| 775 | C38 | Ethinyl estradiol | Dienogest |
| 776 | C39 | Ethinyl estradiol | Dienogest |
| 777 | C40 | Ethinyl estradiol | Dienogest |
| 778 | C41 | Ethinyl estradiol | Dienogest |
| 779 | C42 | Ethinyl estradiol | Dienogest |
| 780 | C43 | Ethinyl estradiol | Dienogest |
| 781 | C44 | Ethinyl estradiol | Dienogest |
| 782 | C45 | Ethinyl estradiol | Dienogest |
| 783 | C46 | Ethinyl estradiol | Dienogest |
| 784 | C47 | Ethinyl estradiol | Dienogest |
| 785 | C48 | Ethinyl estradiol | Dienogest |
| 786 | C49 | Ethinyl estradiol | Dienogest |
| 787 | C50 | Ethinyl estradiol | Dienogest |
| 788 | C51 | Ethinyl estradiol | Dienogest |
| 789 | C52 | Ethinyl estradiol | Dienogest |
| 790 | C53 | Ethinyl estradiol | Dienogest |
| 791 | C54 | Ethinyl estradiol | Dienogest |
| 792 | C55 | Ethinyl estradiol | Dienogest |
| 793 | C56 | Ethinyl estradiol | Dienogest |
| 794 | C57 | Ethinyl estradiol | Dienogest |
| 795 | C58 | Ethinyl estradiol | Dienogest |
| 796 | C59 | Ethinyl estradiol | Dienogest |
| 797 | C60 | Ethinyl estradiol | Dienogest |
| 798 | C61 | Ethinyl estradiol | Dienogest |
| 799 | C62 | Ethinyl estradiol | Dienogest |
| 800 | C63 | Ethinyl estradiol | Dienogest |
| 801 | C64 | Ethinyl estradiol | Dienogest |
| 802 | C65 | Ethinyl estradiol | Dienogest |
| 803 | C66 | Ethinyl estradiol | Dienogest |
| 804 | C67 | Ethinyl estradiol | Dienogest |

### Biological Assays

The utility of the combinations of the present invention can be shown by the following assays. These assays are performed in vitro and in animal models essentially using procedures recognized to show the utility of the present invention.

Rat Carrageenan Foot Pad Edema Test

The carrageenan foot edema test is performed with materials, reagents and procedures essentially as described by Winter, et al., (Proc. Soc. Exp. Biol. Med., 111, 544 (1962)). Male Sprague-Dawley rats are selected in each group so that the average body weight is as close as possible. Rats are fasted with free access to water for over sixteen hours prior to the test. The rats are dosed orally (1 mL) with compounds suspended in vehicle containing 0.5% methylcellulose and 0.025% surfactant, or with vehicle alone. One hour later a subplantar injection of 0.1 mL of 1% solution of carrageenan/sterile 0.9% saline is administered and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Otterness and Bliven, Laboratory Models for Testing NSAIDS, in Non-steroidal Anti-Inflammatory Drugs, (J. Lombardino, ed. 1985)). The % inhibition shows the % decrease from control paw volume determined in this procedure.

Rat Carrageenan-induced Analgesia Test

The analgesia test using rat carrageenan is performed with materials, reagents and procedures essentially as described by Hargreaves, et al., (Pain, 32, 77 (1988)). Male Sprague-Dawley rats are treated as previously described for the Carrageenan Foot Pad Edema test. Three hours after the injection of the carrageenan, the rats are placed in a special plexiglass container with a transparent floor having a high intensity lamp as a radiant heat source, positionable under the floor. After an initial twenty minute period, thermal stimulation is begun on either the injected foot or on the contralateral uninjected foot. A photoelectric cell turns off the lamp and timer when light is interrupted by paw withdrawal. The time until the rat withdraws its foot is then measured. The withdrawal latency in seconds is determined for the control and drug-treated groups, and percent inhibition of the hyperalgesic foot withdrawal determined.

Evaluation of COX-1 and COX-2 Activity in vitro

The compounds of this invention exhibit inhibition in vitro of COX-2. The COX-2 inhibition activity of the compounds of this invention illustrated in the Examples is determined by the following methods.

a. Preparation of Recombinant COX Baculoviruses

A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10 e8) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (10E7-10E8 pfu/ml) stocks of virus are prepared. For large scale production, SF9 insect cells are infected in 10 liter fermentors (0.5×10⁶/ml) with the recombinant baculovirus stock such that the multiplicity of infection is 0.1. After 72 hours the cells are centrifuged and the cell pellet homogenized in Tris/Sucrose (50 mM: 25%, pH 8.0) containing 1% 3-[(3-cholamidopropyl)dimethylammonio]-1-propanesulfonate (CHAPS) . The homogenate is centrifuged at 10,000×G for 30 minutes, and the resultant supernatant is stored at −80° C. before being assayed for COX activity.

b. Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C./room temperature by transferring 40 μl of reaction mix into 160 μl ELISA buffer and 25 μM indomethacin. The PGE2 formed is measured by standard ELISA technology (Cayman Chemical).

The examples herein can be performed by substituting the generically or specifically described reactants and/or operating conditions of this invention for those used in the preceding examples.

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

What is claimed is:

1. A therapeutic combination comprising an amount of a COX-2 inhibitor compound source and an amount of a sex steroid compound wherein the amount of a COX-2 inhibitor compound source and the amount of the sex steroid compound together comprises a dysmenorrhea-effective amount of the compounds.

2. The combination of claim 1 wherein the COX-2 inhibitor source is a COX-2 inhibitor.

3. The combination of claim 2 wherein the COX-2 inhibitor is a tricyclic COX-2 inhibitor.

4. The combination of claim 3 wherein the tricyclic COX-2 inhibitor is selected from the group consisting of a pyrazole COX-2 inhibitor, a furanone COX-2 inhibitor, an isoxazole COX-2 inhibitor, a pyridine COX-2 inhibitor, and a pyridazinone COX-2 inhibitor.

5. The combination of claim 4 wherein the tricyclic COX-2 inhibitor is a pyrazole COX-2 inhibitor.

6. The combination of claim 5 wherein the tricyclic COX-2 inhibitor is celecoxib.

7. The combination of claim 5 wherein the tricyclic COX-2 inhibitor is deracoxib.

8. The combination of claim 4 wherein the tricyclic COX-2 inhibitor is a furanone COX-2 inhibitor.

9. The combination of claim 8 wherein the tricyclic COX-2 inhibitor is rofecoxib.

10. The combination of claim 4 wherein the tricyclic COX-2 inhibitor is an isoxazole COX-2 inhibitor.

11. The combination of claim 10 wherein the tricyclic COX-2 inhibitor is valdecoxib.

12. The combination of claim 4 wherein the tricyclic COX-2 inhibitor is a pyridine COX-2 inhibitor.

13. The combination of claim 12 wherein the tricyclic COX-2 inhibitor is 5-chloro-6′-methyl-3-[4-(methylsulfonyl)phenyl]-2,3′-bipyridine.

14. The combination of claim 4 wherein the tricyclic COX-2 inhibitor is a pyridazinone COX-2 inhibitor.

15. The combination of claim 14 wherein the pyridazinone COX-2 inhibitor is 2-(3,4-difluorophenyl)-4-(3-hydroxy-3-methylbutoxy)-5-[4-(methylsulfonyl)phenyl]-3(2H)-pyridazinone.

16. The combination of claim 2 wherein the COX-2 inhibitor is a benzopyran COX-2 inhibitor.

17. The combination of claim 2 wherein the COX-2 inhibitor is a methane sulfonanilide COX-2 inhibitor.

18. The combination of claim 17 wherein the methane sulfonanilide COX-2 inhibitor is N-(4-nitro-2-cyclohexyloxyphenyl)methanesulfonamide.

19. The combination of claim 1 wherein the COX-2 inhibitor source is a prodrug of a COX-2 inhibitor.

20. The combination of claim 19 wherein the prodrug of the COX-2 inhibitor is parecoxib.

21. The combination of claim 1 wherein the sex steroid compound is a progestin sex steroid.

22. The combination of claim 1 wherein the sex steroid compound is an estrogen sex steroid.

23. The combination of claim 22 wherein the sex steroid compound further comprises a progestin sex steroid.

24. The combination of claim 23 wherein the sex steroid compound comprises an amount of an estrogen sex steroid and an amount of a progestin sex steroid wherein the amount of the estrogen sex steroid and the amount of the progestin sex steroid together comprise a menstrual cycle controlling-effective amount of the compounds.

25. The combination of claim 24 wherein the estrogen sex steroid is ethinyl estradiol.

26. The combination of claim 24 wherein the progestin sex steroid is selected from the group consisting of levonorgestrel, norethindrone acetate, norgestimate, ethynodiol acetate, desogestrel, norgestrel and norethindrone.

27. The combination of claim 26 wherein the progestin sex steroid is levonorgestrel.

28. The combination of claim 26 wherein the progestin sex steroid is norethindrone acetate.

29. The combination of claim 26 wherein the progestin sex steroid is norgestimate.

30. The combination of claim 26 wherein the progestin sex steroid is ethynodiol acetate.

31. The combination of claim 26 wherein the progestin sex steroid is desogestrel.

32. The combination of claim 26 wherein the progestin sex steroid is norgestrel.

33. The combination of claim 26 wherein the progestin sex steroid is norethindrone.

34. The combination of claim 1 wherein the COX-2 inhibitor compound source and the sex steroid compound are present in a single composition.

35. A combination therapy method for the treatment or prophylaxis of dysmenorrhea in a patient in need thereof, comprising:
administering to the patient an amount of a COX-2 inhibitor compound source and administering to the patient an amount of a sex steroid compound wherein the amount of the COX-2 inhibitor compound source and the amount of the sex steroid compound together comprise a dysmenorrhea-effective amount of the compounds

36. The combination therapy method of claim 35 wherein the COX-2 inhibitor source is a COX-2 inhibitor.

37. The combination therapy method of claim 36 wherein the COX-2 inhibitor compound is celecoxib.

38. The combination therapy method of claim 36 wherein the COX-2 inhibitor compound is rofecoxib.

39. The combination therapy method of claim 36 wherein the COX-2 inhibitor compound is valdecoxib.

40. The combination therapy method of claim 36 wherein the COX-2 inhibitor compound is deracoxib.

41. The combination therapy method of claim 36 wherein the COX-2 inhibitor compound is 5-chloro-6′-methyl-3-[4-(methylsulfonyl)phenyl]-2,3′-bipyridine.

42. The combination therapy method of claim 36 wherein the COX-2 inhibitor compound is N-(4-nitro-2-phenoxyphenyl)methanesulfonamide.

43. The combination therapy method of claim 36 wherein the COX-2 inhibitor compound is 2-(3,4-difluorophenyl)-4-(3-hydroxy-3-methylbutoxy)-5-[4-(methylsulfonyl)phenyl]-3(2H)-pyridazinone.

44. The combination therapy method of claim 35 wherein the COX-2 inhibitor source is a prodrug of a COX-2 inhibitor.

45. The combination therapy method of claim 44 wherein the prodrug of the COX-2 inhibitor is parecoxib.

46. The combination therapy method of claim 35 wherein the sex steroid compound comprises an amount of an estrogen sex steroid and an amount of a progestin sex steroid wherein the amount of the estrogen sex steroid and the amount of the progestin sex steroid together comprise a menstrual cycle controlling-effective amount of the compounds.

47. The combination therapy method of claim 46 wherein the estrogen sex steroid is ethinyl estradiol.

48. The combination therapy method of claim 46 wherein the progestin sex steroid is selected from the group consisting of levonorgestrel, norethindrone acetate, norgestimate, ethynodiol acetate, desogestrel, norgestrel and norethindrone.

49. The combination therapy method of claim 48 wherein the progestin sex steroid is levonorgestrel.

50. The combination therapy method of claim 48 wherein the progestin sex steroid is norethindrone acetate.

51. The combination therapy method of claim 48 wherein the progestin sex steroid is norgestimate.

52. The combination therapy method of claim 48 wherein the progestin sex steroid is ethynodiol acetate.

53. The combination therapy method of claim 48 wherein the progestin sex steroid is desogestrel.

54. The combination therapy method of claim 48 wherein the progestin sex steroid is norgestrel.

55. The combination therapy method of claim 48 wherein the progestin sex steroid is norethindrone.
